SEQUENCE LISTING (4 sequences)

SEQ ID NO: 1 - DNA, 20 bp, Artificial Sequence
  Synthetic construct Ova-F primer
aagcaggcagagaggtggta

SEQ ID NO: 2 - DNA, 20 bp, Artificial Sequence
  Synthetic construct Ova-R primer
gaatggatggtcagccctaa

SEQ ID NO: 3 - DNA, 22 bp, Artificial Sequence
  Synthetic construct beta-actin-F primer
aagatctggcaccacaccttct

SEQ ID NO: 4 - DNA, 20 bp, Artificial Sequence
  Synthetic construct beta-actin-R primer
ttttcacggttggccttagg

(19) United States
(12) Patent Application Publication
(10) Pub. No.: US 2022/0213505 A1
(43) Pub. Date: Jul. 7, 2022

(54) INDUCING IMMUNE TOLERANCE BY rAAV VECTORS

(71) Applicants: UNIVERSITÉ DE PARIS, Paris (FR); GENETHON, Evry (FR); CENTRE NATIONAL DE LA RECHERCHE SCIENTIFIQUE, Paris (FR); INSTITUT NATIONAL DE LA SANTE ET DE LA RECHERCHE MEDICALE, Paris (FR)

(72) Inventors: Federico MINGOZZI, Philadelphia, PA (US); Jean DAVOUST, Saint-Germain-en-Laye (FR); Laurent BARTOLO, Philadelphia, PA (US)

(21) Appl. No.: 17/610,840

(22) PCT Filed: May 15, 2020
(86) PCT No.: PCT/EP2020/063733

§ 371 Date: Nov. 12, 2021

(30) Foreign Application Priority Data
May 15, 2019 (EP) 19305623.1

(51) Int. Cl. C12N 15/86; A61P 21/00; A61P 37/06; A61K 48/00

(52) U.S. Cl. CPC C12N 15/86; A61P 21/00; A61P 37/06; C12N 2830/008; A61K 48/0083; C12N 2750/14143; C12N 2750/14171; A61K 48/0058

(57) ABSTRACT

A combination of two recombinant adeno-associated viral (rAAV) vectors comprising in the first a capsid and a cassette comprising a 5′ ITR sequence, a liver-specific promotor, a nucleic acid sequence coding for a transgene of interest useful to be tolerated by the immune system and a poly A chain and in the second a capsid and a cassette comprising a 5′ ITR sequence, a promotor specific for a tissue of interest, a nucleic acid sequence corresponding to the nucleic acid sequence inserted into the cassette of the first rAAV vector, a transmembrane sequence, a poly A chain, wherein the nucleic acid sequences is administered towards to the tissue of interest, is disclosed. Said combination can be used in inducing an immune tolerance to a protein product encoded, and so be used as a drug in a subject, particularly for treating muscular dystrophies. Pharmaceutical compositions, kits and methods are also disclosed.

### FIELD OF THE INVENTION

The present invention relates to the field of gene therapy. Particularly, the invention relates to a combination of two recombinant adeno-associated viral (rAAV) vectors, the first comprising a capsid and a cassette comprising a 5′ ITR sequence, a liver-specific promotor, a nucleic acid sequence of interest useful to be tolerated by the immune system and a poly A chain and the second comprising a capsid and a cassette comprising a 5′ ITR sequence, a promotor specific for a tissue of interest, a nucleic acid sequence corresponding to the nucleic acid sequence inserted into the cassette of the first rAAV vector, a poly A chain, wherein the nucleic acid sequence is administered towards the tissue of interest. Said combination can be used in inducing an immune tolerance to a protein product encoded and translated from said nucleic acid sequence delivered to the said tissue of interest and so, be used as a drug in a subject, particularly for treating muscular dystrophies. The invention also relates to pharmaceutical composition, kits and a method to prevent a risk of rejection of said nucleic acid sequence.

### BACKGROUND OF THE INVENTION

Recombinant adeno associated virus (rAAV) vectors are widely used for gene transfer applications in peripheral tissues and proved to safely deliver a variety of therapeutic transgenes to treat affections of monogenic origin such as neuromuscular, ocular, neurodegenerative and hemophilia disorders. These gene reparative medicine applications rely on successful engraftment of a defined transgene in the tissue of interest. Compared to classical tissue engraftment procedures implying the transfer of allogenic cells and MHC components, rAAV gene transfer raises specific concerns related to the immunogenicity of rAAV capsids and the processing and recognition of a newly expressed transgene by the host immune system. Preexisting anti-capsid antibody responses observed for AAV serotypes mostly prevalent in humans, can impair treatment ineffectiveness, advocating for the use of other rAAV serotypes, engineered capsids and immunosuppression procedures. Of equal importance, cytotoxic T cell (CTL) responses to the capsid were encountered in human liver clinical trials, representing an important concern, currently handled with transient immunosuppression regimens.

In parallel to anti-capsid responses, immune responses to newly expressed transgenes depends on multiple factors intrinsic to the recipient such as the mutational genotype of the host, the route of injection, the promotor being used, the rAAV dose and the initial inflammatory and metabolic disorder status present in the tissue to be injected. The occurrence of preexisting immune responses to the transgene represents also challenging issue. In the case of hemophilia B patients, preexisting humoral responses against coagulation factor IX (FIX) have been observed in humans in relation with protein replacement therapies. Animal studies have also evidenced immune responses to FIX gene transfer especially in FIX KO animals, in which the FIX transgene is considered as a foreign antigen by the immune system. This points out the important role of the genetic background of the host in the generation of transgene-specific T cells and multiple genetic components concur in defining the immune response to a given transgene. As an example, cytotoxic CD8⁺T cell responses were observed after human FIX gene transfer in C57/BI6 mice but were absent in other mouse strains. The target tissue itself is also an important factor influencing the outcome of immune responses after gene transfer and rAAV muscle targeting is known to be highly immunogenic using model transgenes but also with cell associated transgene delivery to treat monogenic muscle disorders. Of note, the presence of preexisting circulating T cell immunity to dystrophin was observed in a sizable proportion of Duchenne muscular dystrophy patients, possibly driving the immune-mediated rejection of the microdystrophyn transgene delivered intramuscularly with AAV vectors and advocating for transgene-specific immunomodulation.

Harnessing the tolerogenic properties of the liver, reports showed that expression of an allogenic MHC component in the liver allowed successful engraftment of a transgenic skin graft bearing the same alto MHC antigen. Likewise, rAAV-mediated liver targeting proved to be safe and efficient in hemophilia mouse models with a human FIX transgene or using a variety of transgenes and rAAV serotypes. rAAV FIX gene transfer in liver induced no noticeable humoral or cellular responses against the FIX transgene, which is a typically secreted protein accessible throughout the body, and this tolerance state was conserved after secondary FIX immunization in rodents. Moreover, recent mouse studies showed that rAAV FIX liver targeting can override preexisting anti-transgene humoral immunity. Regarding CTL responses to the transgene, the capacity of liver transduction to control the fate of transgene-specific TCR transgenic CD8⁺T cells was initially unraveled by the team of Bertolino (Bowen et al. 2004) who showed that the site of primary T cell activation dictates the balance between intrahepatic tolerance and immunity, a result confirmed with other models. The fraction of transduced hepatocytes was critical to induce immune tolerance after hepatic gene transfer, leading to the acquisition of an exhausted phenotype for TCR transgenic CD8⁺T cells.

Considering the fact that transduction of multiple tissues apart the liver, leads to the generation of prominent humoral and cellular immune responses to the transgene, there is a need for a novel vector transduction protocol able to harness immune tolerance and in particular that of humoral and cellular responses comprising CD8⁺ and CD4⁺T cells to cell-associated transgene. Moreover, there is an additional need to nullify the adverse effect of preexisting CD8⁺ and CD4⁺T cell immunity to the transgene, to avoid when present, the reactivation of CD4⁺ and CD8⁺ memory T cells directed to the transgene. Thus, an alternative and/or improved method, especially based on rAAV vectors, is needed for successful gene therapy to avoid adverse humoral and cellular immune responses to cell-associated transgene delivered in multiple tissues.

### SUMMARY OF THE INVENTION

The invention aims to remedy the disadvantages of prior art. In particular the invention proposes a combination of recombinant adeno-associated viral (rAAV) vectors for use as a drug in inducing an immune tolerance to the protein product encoded and translated from a nucleic acid sequence present in the cassette of the said recombinant adeno-associated viral (rAAV) vectors in a subject.

In a first aspect, the invention relates to combination of

i. a first recombinant adeno-associated viral (rAAV) vector comprising a capsid and a cassette comprising a 5′ ITR sequence, a liver-specific promotor, a nucleic acid sequence coding for a protein product to be tolerated by the immune system, a poly A chain, and

ii. a second recombinant adeno-associated viral (rAAV) vector comprising a capsid and a cassette comprising a 5′ ITR sequence, a muscle-specific promotor, a nucleic acid sequence coding for the protein product to be tolerated by the immune system of the first recombinant adeno-associated viral vector (i), a poly A chain,

for use in treating a muscular dystrophy, preferably a monogenic muscle disorder, in a subject, wherein the first rAAV is administered to target the liver and the second rAAV is administered to target muscle tissues.

The combination of the invention is particularly suitable to provide immune tolerance for a cell-associated protein product, for example cytosolic or membrane associated protein like e. g. transmembrane proteins, that are not secreted. Accordingly, in a particular aspect, the invention relates to a combination as exposed above, wherein the protein product to be tolerated is a cell-associated protein product, for example cytosolic or membrane associated protein like e. g. transmembrane proteins.

As shown by the inventors of the present application, the combination according to the invention is particularly effective in providing immune tolerance toward immunogenic proteins. Then, in a particular embodiment of the combination of the invention, the nucleic acid sequence coding for a protein product to be tolerated by the immune system codes for a protein product comprising an epitope recognized by T-cells or B-cells.

Combination of the invention allows to eliminate or attenuate both the humoral and cellular immune response, and particularly CD8⁺ immune response which is of particular interest in treating muscular dystrophies. Hence in a further embodiment, the invention relates to a combination as stated above, for use in treating a muscular dystrophy comprising eliminating or attenuating the occurrence of cellular and humoral immune responses to the protein product, thereby allowing said protein product to be tolerated by the immune system and/or its expression in muscle; In a more particular embodiment the invention relates to a combination as stated above, for use in treating a muscular dystrophy comprising inducing cytotoxic CD8⁺ T-cell tolerance.

As exemplified, dual muscle liver transduction using the combination of the invention, allows to induce immune tolerance in both subjects that either do not present immune response for the protein product of the combination of the invention, or subjects that present a preexisting immune response for the protein product expressed by the combination. Said preexisting response can be due for example to a previous gene replacement therapy to which the subject has been applied. In a particular object, the combination is thus administered to a subject which presents a preexisting immunity towards the protein product to be tolerated by the immune system.

In other optional aspects of the combination for use according to the invention:
the liver-specific promotor of the first rAAV vector (i) is a hepatocyte-specific promotor (hAAT),
the capsid of the first and second rAAV vector is selected from the group consisting in AAV1, AAV2, AAV3, AAV4, AAV5, AAV6, AAV7, AAV8, AAV9, AAV10, AAV11 or any combination thereof,
the capsid of the first rAAV vector is an AAV8 capsid,
the protein product to be tolerated by the immune system is a muscle specific protein or a neuromuscular protein,
the nucleic acid sequence coding for a protein product to be tolerated by the immune system is coding for a peptide selected from the sequence of microdystrophin constructs, Emerin, Lamin A/C, Spectrin repeat containing, nuclear envelope 1 (nesprin 1), Spectrin repeat containing, nuclear envelope 2 (nesprin 2), Transmembrane protein 43, Torsin A interacting protein 1, Double homeobox 4, Structural maintenance of chromosomes flexible hinge domain containing 1, Polymerase I and transcript release factor(M), Myotilin, Caveolin 3, HSP-40 homologue, subfamily B, number 6, Desmin, Transportin 3, Heterogeneous nuclear ribonucleoprotein D-like, Calpain 3, Dysferlin, Gamma sarcoglycan, Alpha sarcoglycan, Beta sarcoglycan, Delta-sarcoglycan, Telethonin, Tripartite motif-containing 32, Fukutin-related protein, Titin, Protein-O-mannosyltransferase 1, Anoctamin 5, Protein-O-mannosyltransferase 2, O-linked mannose beta1,2-N-acetylglucosaminyltransferase, Dystroglycan1, plectin, Desmin, trafficking protein particle complex 11, GDP-mannose pyrophosphorylase B, Isoprenoid synthase domain containing, Acid alpha-glucosidase preproprotein, LIM and senescent cell antigen-like domains 2, blood vessel epicardial substance, Torsin A interacting protein 1, Protein 0-Glucosyltransferase 1, Dolichyl-phosphate mannosyltransferase polypeptide 3, Valosin-containing protein, plectin,
the first rAAV vector is administered intravenously and the second rAAV vector is administered intramuscularly to the subject, or
the first rAAV vector is administered before the second rAAV vector, preferably one week, even more preferably one month before the second rAAV vector.

In a particular embodiment of the invention, the combination in any of the embodiments as stated above, for use in treating Duchenne Muscular Dystrophy (DMD) and wherein the protein product to be tolerated by the immune system is a microdystrophin construct.

Another object of the invention is a pharmaceutical composition comprising the combination of the invention in any of its embodiments as state above for use in treating a muscular dystrophy, preferably from a monogenic muscle disorder, more preferably from the Duchenne muscular dystrophy.

In another aspect, the invention relates to a combination comprising:

i. a first recombinant adeno-associated viral vector comprising a capsid and a cassette comprising a 5′ ITR sequence, a liver-specific promotor, a nucleic acid sequence of interest useful to be tolerated by the immune system, a poly A chain, wherein the nucleic acid sequences is administered to be deliver toward the liver, and

ii. a second recombinant adeno-associated viral vector comprising a capsid and a cassette comprising a 5′ ITR sequence, a promotor specific for a tissue of interest, a nucleic acid sequence corresponding to the nucleic acid sequence inserted into the cassette of the first recombinant adeno-associated viral vector, a transmembrane sequence, a poly A chain, wherein the nucleic acid sequence is administered towards the tissue of interest,

for use as a drug in inducing an immune tolerance to a protein product encoded and translated from said nucleic acid sequence delivered to the said tissue of interest in a subject.

Said two recombinant adeno-associated viral vectors comprise a cassette comprising a nucleic acid sequence coding for a cell-associated protein product, preferably it is a transmembrane protein product.

In a preferred embodiment, the nucleic acid sequence of interest inserted in the cassette of the two recombinant adeno-associated viral vectors is coding for a protein product comprising an epitope recognized by T cells or B-cells. Alternately, the nucleic acid sequence of interest is coding for a muscle-associated protein, preferably a membrane protein. In another embodiment, the nucleic acid sequence of interest is coding for a muscle specific protein or neuromuscular protein, preferably the nucleic acid sequence of interest is coding for the sequence of microdystrophin constructs. In another preferred but non limited embodiment, the promotor specific for a tissue of interest in the second vector (ii) is a muscle-specific promotor.

Advantageously, the combination of the two-recombinant adeno-associated viral (rAAV) vectors according to the invention is administered to the subject after a prior immunization of CD4, CD8 and/or B lymphocytes. Preferably, the combination is administered to the subject exhibiting a noticeable level of immunization of CD4, CD8 and/or B lymphocytes directed toward the protein product encoded by said nucleic acid sequence.

In another preferred embodiment, the promoter of the first recombinant adeno-associated viral vector is a liver-specific promotor, preferably a hepatocyte-specific promotor (hAAT). The capsid of the first, second or both recombinant adeno-associated viral vector according to the invention is selected from the group consisting in AAV1, AAV2, AAV3, AAV4, AAV5, AAV6, AAV7, AAV8, AAV9, AAV10, AAV11 and any combination. In a more preferred embodiment, the capsid of the first rAAV delivered toward the liver is an AAV8 capsid.

In a preferred embodiment, the combination of the present invention is useful to be use for treating Duchenne Muscular Dystrophy (DMD). In another preferred embodiment, the second recombinant adeno-associated viral vector comprising a nucleic acid sequence of interest is for use in the treatment of Duchenne Muscular Dystrophy.

In a preferred embodiment, the combination is administered simultaneously or sequentially.

In a second aspect, the invention relates to a pharmaceutical composition comprising a first recombinant adeno-associated viral vector as described below and a second recombinant adeno-associated viral vector comprising a capsid and a cassette comprising a 5′ ITR sequence, a promotor specific for a tissue of interest, the nucleic acid sequence corresponding to the nucleic acid sequence inserted into the cassette of the first recombinant adeno-associated viral vector, a transmembrane sequence, a poly A chain, wherein the nucleic acid sequence is administered towards the tissue of interest, for use as a drug in inducing an immune tolerance to said nucleic acid sequence delivered to the said tissue of interest in a subject.

In a preferred embodiment, the pharmaceutical composition is administered simultaneously or sequentially. In another embodiment, the pharmaceutical composition can be administered twice or thrice, as many times as desired and/or repetitively. Advantageously, the pharmaceutical composition is administered intravenously or intramuscularly.

In a more preferred embodiment, the pharmaceutical composition is useful in gene therapy, preferably in muscular dystrophies, preferably a monogenic muscle disorder, more preferably the Duchenne Muscular Dystrophy. Alternately, the pharmaceutical composition is useful in auto-immune disorders, using the said nucleic acid sequence coding for the protein targeted by the auto-immune responses.

### LEGEND OF DRAWING

FIG. 1. rAAV Constructs.

The mOVA construct comprises the leader peptide from the H-2Kb gene (LS), the full-length OVA cDNA including the MHC I and MHC II epitope, OVA257 and OVA323 respectively, the H-2db transmembrane sequence (TM) followed by a STOP codon and a poly A chain (pA). The mOVA-GFP construct comprises the leader peptide from the H-2Kb gene (LS), the full-length OVA cDNA, the H-2db transmembrane sequence (TM) and the full-length EGFP cDNA. The muscle targeting construct (A) contains the muscle-specific promotor SPc5-12 and two ITR (Inverted Terminal Repeat) sequences for encapsulation in rAAV1, which have a strong tropism for muscle. The liver targeting construct (B) contains the liver-specific promotor hAAT and two ITR sequences for encapsulation in rAAV8, which have a strong tropism for liver. The liver targeting construct (C) contains the liver-specific promotor hAAT and two ITR sequences for encapsulation in rAAV8 and the full hFIX transgene cassette.

FIG. 2. Transgene-Specific Immune Tolerance in Muscle is Imposed by Concurrent Liver targeting.

Male C57/BI6 mice were injected in the left tibialis anterior muscle with 10⁹ viral genomes (vg) of rAAV1 encoding mOVA under the muscle-specific SPc5-12 promotor and injected i.v. with 10¹⁰ vg rAAV8 encoding mOVA under the liver-specific promotor hAAT. Experimental conditions listed correspond to rAAV1/mOVA i.m. injection and to simultaneous injections of rAAV1/mOVA i.m. and rAAV8/mOVA i.v. Lymphocytes were extracted from blood at day 14 and 28 to analyze OVA-specific CD8⁺T cells by Kb/OVA257 tetramer staining and cytometry. (A) Representative dot plots at d28 and (B) frequencies of CD8⁺ Kb/OVA257 tetramer⁺ (Tetramer⁺) in blood gated on CD8⁺ T-cells. (C) Concentration of anti-OVA IgG relative to a control serum in arbitrary unit (AU). (D) RT-qPCR performed in muscle at day 29 in the experimental conditions listed. RT-qPCR results are expressed relatively to OVA RNA expression in the “i.m.+i.v.” group (cf. materials and methods). Each dot represents an individual animal, mean±SEM (n=9 mice per group, pooled from three independent experiments). **p<0.01, ****p<0.0001 (Mann-Whitney test).

FIG. 3. Transgene-Specific CD8⁺T Cell Tolerance is Established in Muscle, Long after liver transduction.

Male C57/BI6 mice were injected i.v. with 10¹⁰ vg of rAAV8/mOVA at day −28 or −7 or none (control group). At day 0, mice were injected in the left tibialis anterior muscle with 1×10¹⁰ vg of rAAV1/mOVA i.m. Blood was collected at d14 and 28 and mice euthanized at day 29 to collect injected muscle and liver. (A) Time line of the experiment. (B) Frequencies of CD8⁺ Kb/OVA257 Tetramer⁺ (Tetramer⁺) gated on CD8⁺T cells assessed at d28 in the three experimental conditions listed. (C) RT-qPCR performed in muscle at day 29 in the three experimental conditions listed. RT-qPCR results are expressed relatively to OVA RNA expression in the “i.m.+i.v. d-7” group. Each dot represents an individual animal, mean±SEM (n=6 mice per group, pooled from two independent experiments). **p<0.01 (Mann-Whitney test).

FIG. 4. Transgene-Specific CD8⁺T Cell Tolerance Occurs in Muscle Despite Prior immunization.

Male C57/BI6 mice were immunized or not with OVA emulsified in IFA (OVA/IFA) by tail base injection at d0, then injected with the indicating rAAV at d14. Blood was collected at d28 and mice euthanized at day 29 to collect spleen and injected muscle. The rAAV1/mOVA i.m. and the rAAV8/mOVA i.v. injections were performed as described in FIG. 2. The rAAV1/mOVA i.m. injection was performed with 10¹⁰ vg to refine the analysis of T cell populations. Lymphocytes were extracted from spleen to perform Kb/OVA257 tetramer staining. (A) Frequencies of CD8⁺ Kb/OVA257 Tetramer⁺ (Tetramer⁺) gated on CD8⁺T cells in spleen. (B) Quantities of anti-OVA IgG relative to a control serum in arbitrary unit (AU). (C) RT-qPCR performed in muscle at day 29 in the four experimental conditions listed. RT-qPCR results are expressed relatively to OVA RNA expression in the “i.m.+i.v.” no immunized group. Each dot represents an individual animal, mean±SEM (n=9 mice per group pooled from three independent experiments). **p<0.01, ***p<0.001, ****p<0.0001 (Mann-Whitney test).

FIG. 5. Residual OVA-Specific CD8⁺T Cells Arbor a PD-1^{hi} Phenotype after Prior immunization and tolerance induction.

Male C57/BI6 mice were immunized or not with OVA or OVA257 emulsified in IFA (OVA/IFA or OVA257/IFA) and injected as described in FIG. 4 (10¹⁰vg of rAAV1-mOVA i.m. and rAAV8-mOVA i.v.). (A) Representative dot plots of CD8⁺ Kb/OVA257 Tetramer⁺ (Tetramer⁺) and PD-1⁺T cells gated on CD8⁺ CD44^{hi}T cells in spleen assessed at d28 in the four experimental conditions listed. (B) Frequencies of CD8⁺ Kb/OVA257 Tetramer⁺ (Tetramer⁺) T cells gated on CD8⁺ CD44^{hi}T cells in spleen assessed at d28 in the six experimental conditions listed.(C) MFI of expression levels of PD-1 (upper panel), CD44 (middle panel) and CD8 (lower panel) gated on CD8⁺ CD44+ Tetramer⁺T cells after OVA/IFA or OVA257/IFA immunization. Each dot represents an individual animal, mean±SEM (n=6 mice per group, pooled from two independent experiments). *p<0.05 and **p<0.01 (Mann-Whitney test).

FIG. 6. Lack of IFNγ Production in Residual OVA-Specific PD-1^{hi} CD8⁺T Cells

Splenocytes from male C57/BI6 mice immunized or not with OVA or OVA257 emulsified in IFA (OVA/IFA or OVA257/IFA) from the experiment presented in FIG. 5 were stimulated 4 hours in vitro with OVA257 peptide and processed for intracellular staining. (A) Representative dot plots of PD-1⁺ and IFNγ⁺ splenocytes gated on CD8⁺T cell populations after in vitro stimulation with OVA257 peptide. (B) Frequencies of INFy⁺ producing cells gated on CD8⁺T cells in spleen after in vitro stimulation with OVA257 peptide. (C) RT-qPCR performed in muscle at day 29, in the four experimental conditions listed. RT-qPCR results are expressed relatively to OVA RNA expression in the “i.m.+i.v.” no immunized group. Each dot represents an individual animal, mean±SEM (n=6 mice per group, pooled from two independent experiments). **p<0.01, ns p>0.05 (Mann-Whitney test).

FIG. 7. Transgene-Specific CD8⁺T Cell Tolerance is Established Despite CD4⁺T Cell immunization.

Male C57/BI6 mice were immunized or not with the OVA323 peptide (MHCII epitope) emulsified in IFA (OVA323/IFA) and injected as described in FIG. 4 (10¹⁰ vg of rAAV1-mOVA i.m. and rAAV8-mOVA i.v.). Lymphocytes were extracted from spleen to perform CD8⁺T cell Kb/OVA257 tetramer and intracellular INFy staining. (A) Frequencies of INFy⁺ gated on CD4+CD44^{hi}T cells in spleen. (B) Quantities of anti-OVA IgG relative to a control serum in arbitrary unit (AU). (C) Frequencies of CD8⁺ Kb/OVA257 Tetramer⁺ (Tetramer⁺) gated on CD8⁺ CD44^{hi}T cells in spleen. (D) RT-qPCR performed in muscle at day 29 in the four experimental conditions listed. RT-qPCR results are expressed relatively to OVA RNA expression in the “i.m.+i.v.” no immunized group. Each dot represents an individual animal, mean±SEM (n=6-9 mice per group, pooled from two to three independent experiments). ns p>0.05,**p<0.01, ****p<0.0001 (Mann-Whitney test).

FIG. 8. Lack of Tolerance after i.m. And i.v. Disparate Transgene Injections

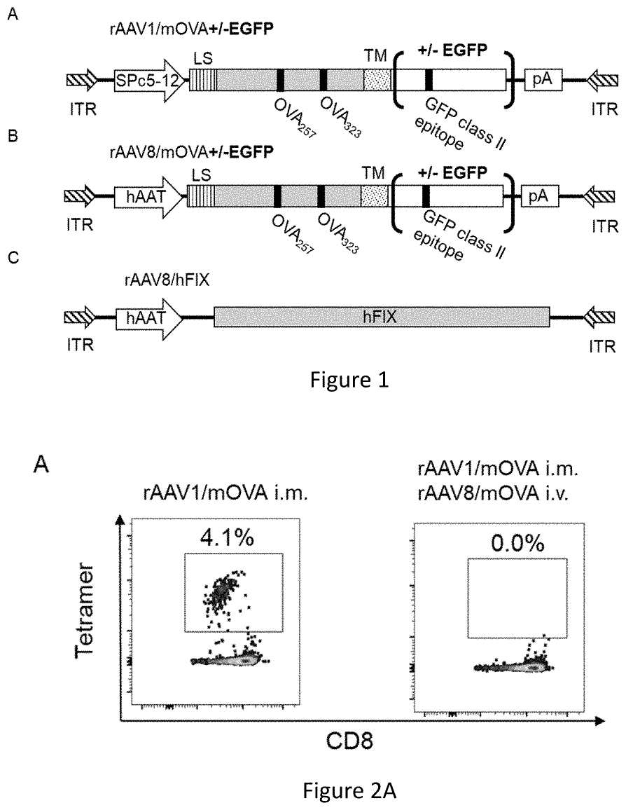

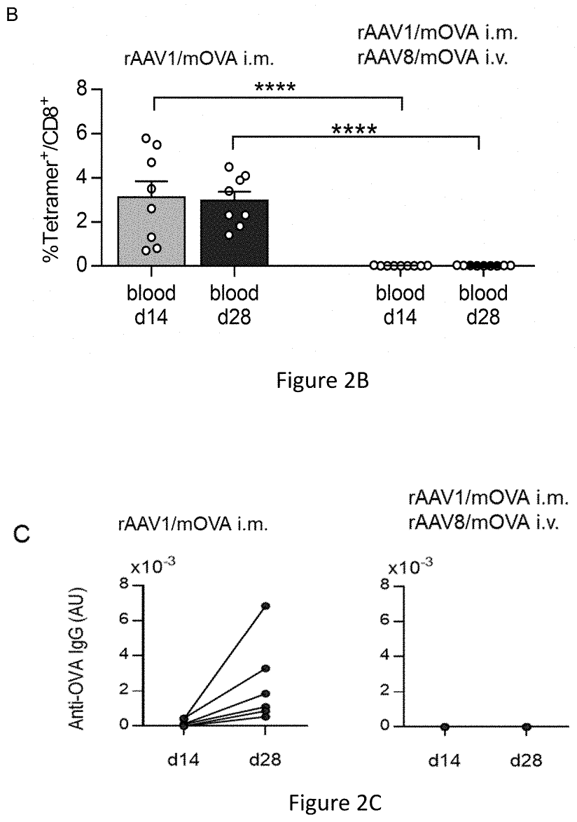

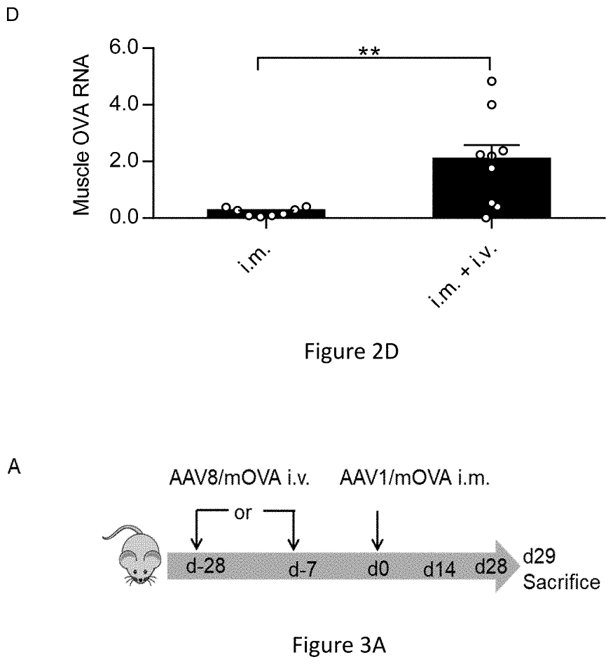

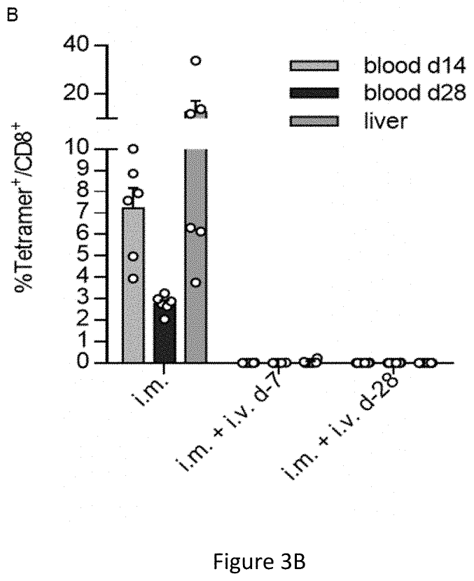

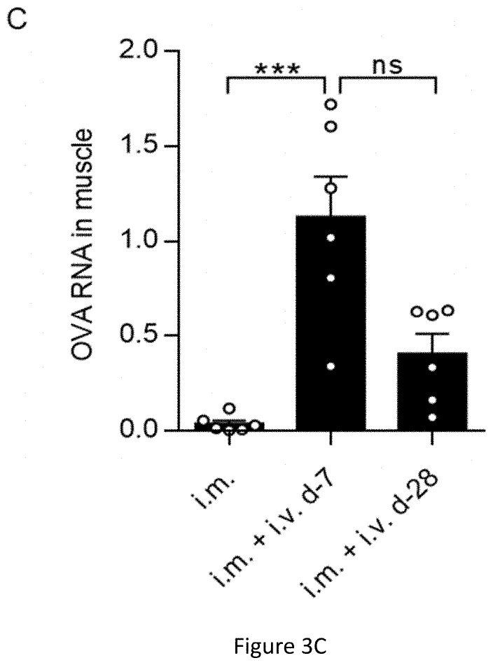

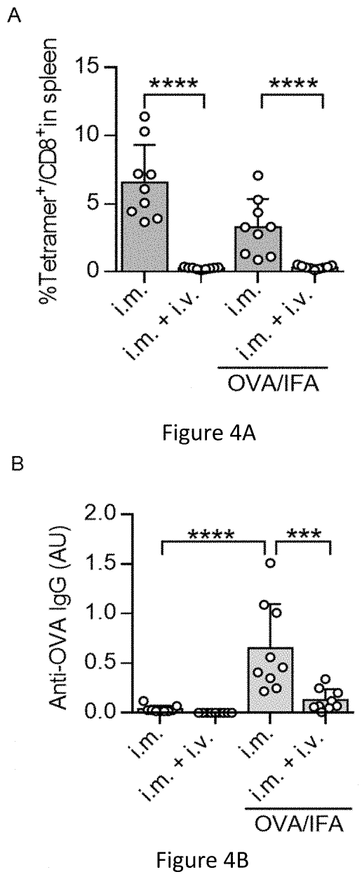

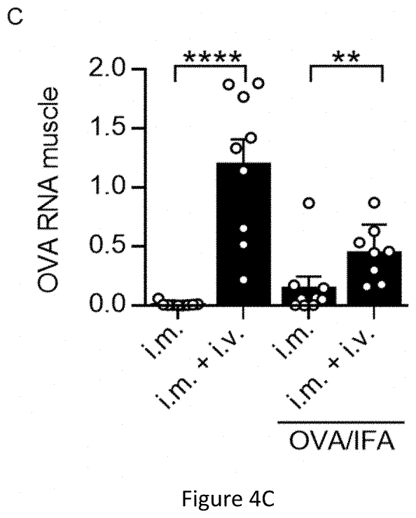

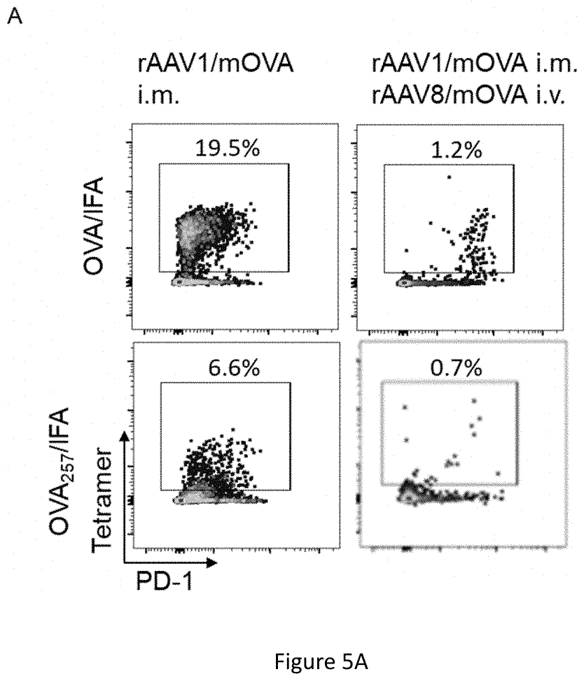

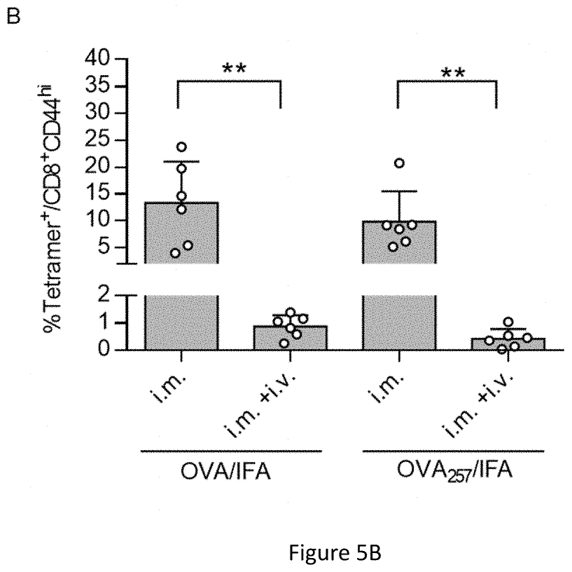

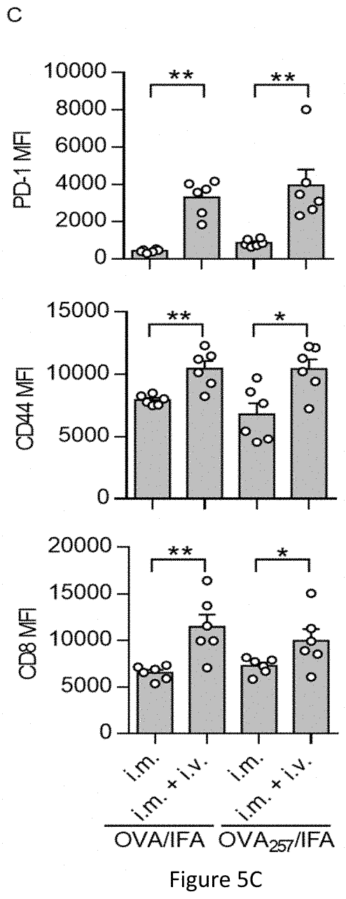

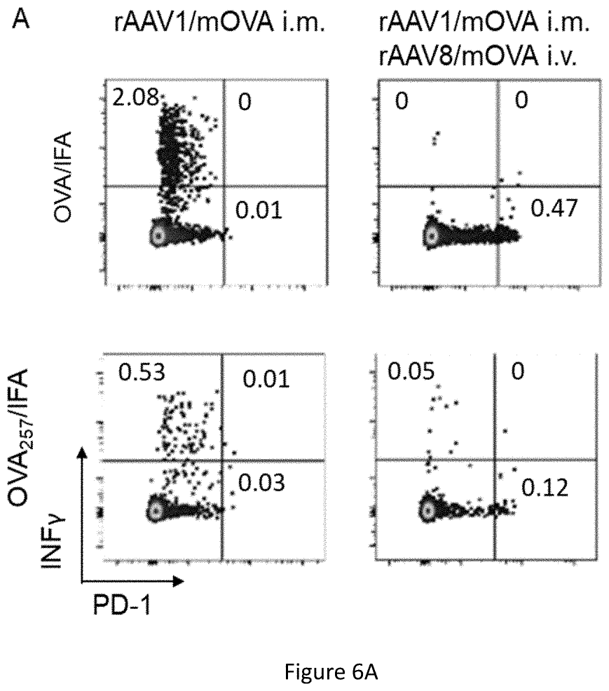

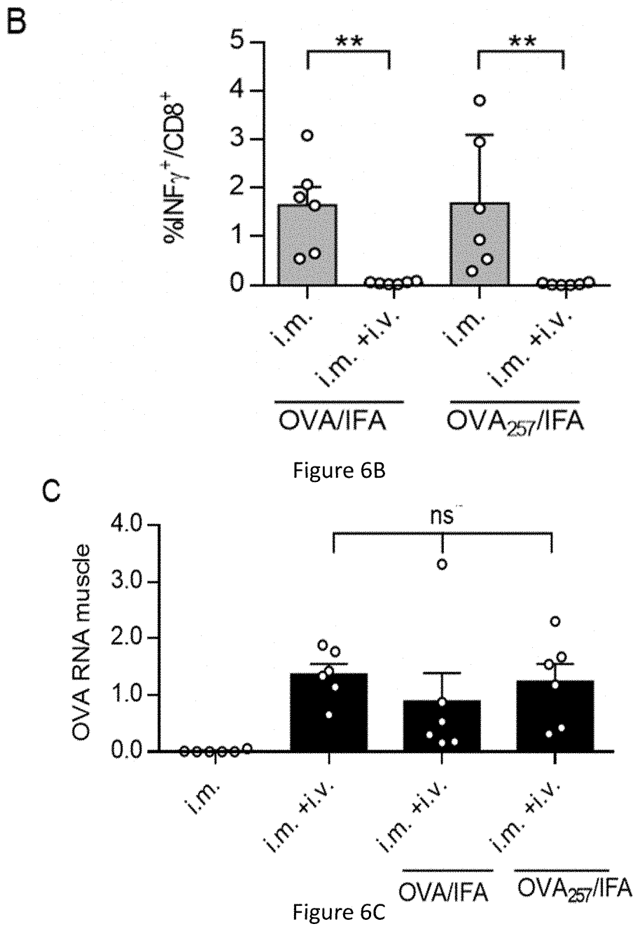

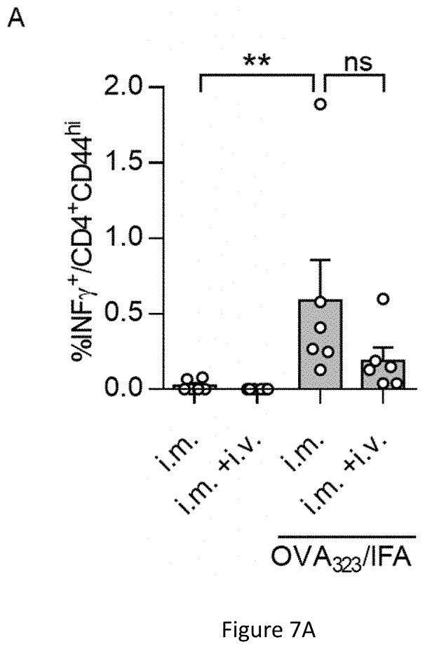

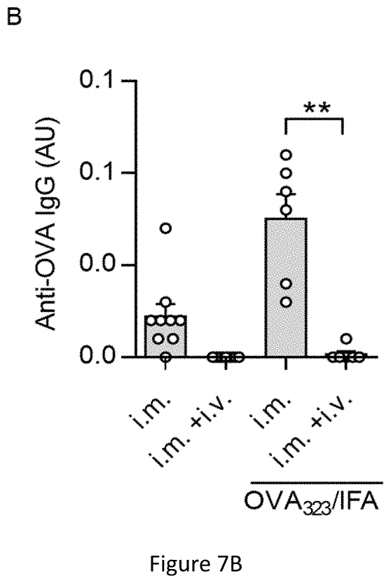

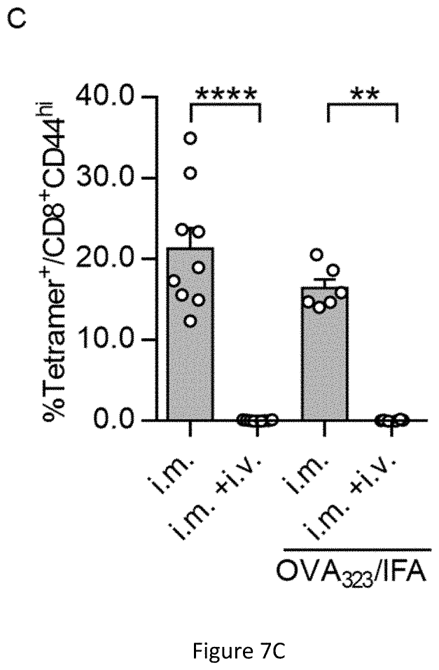

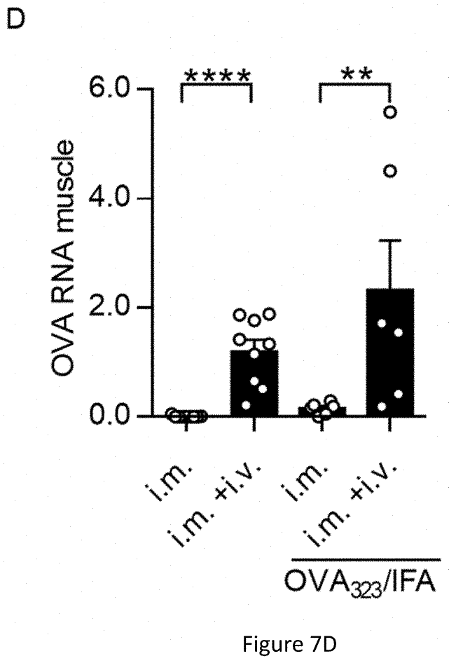

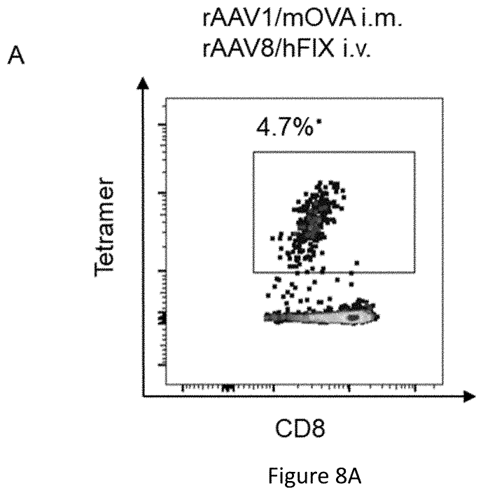

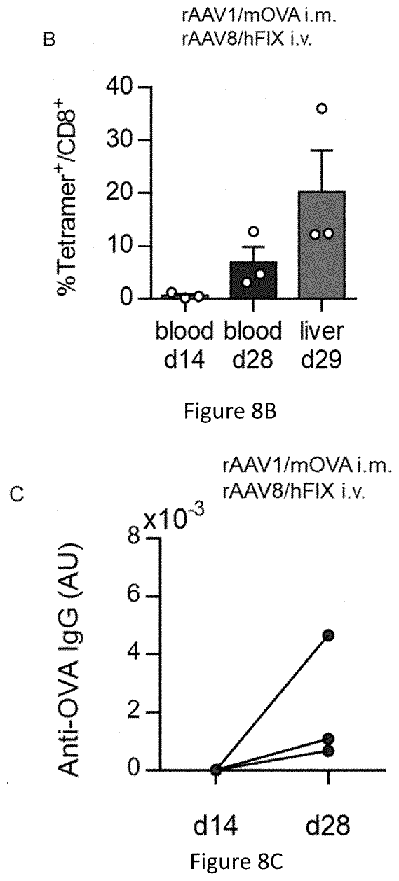

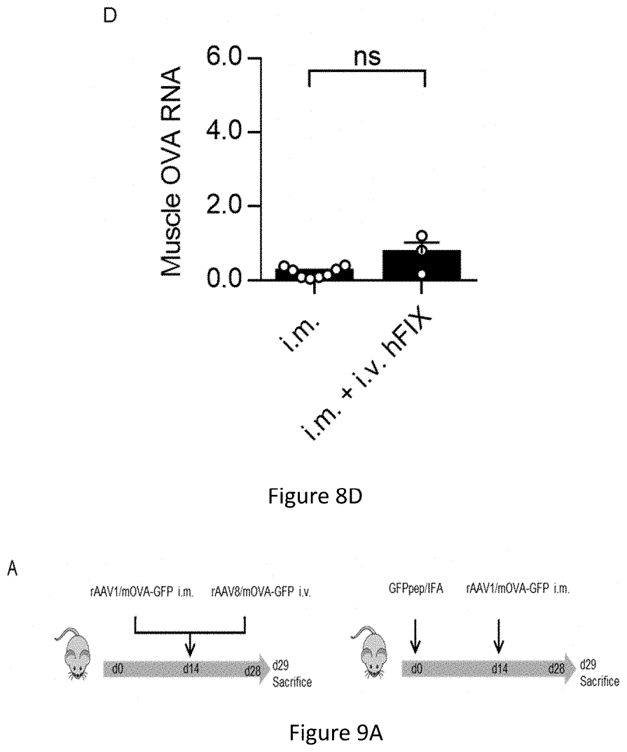

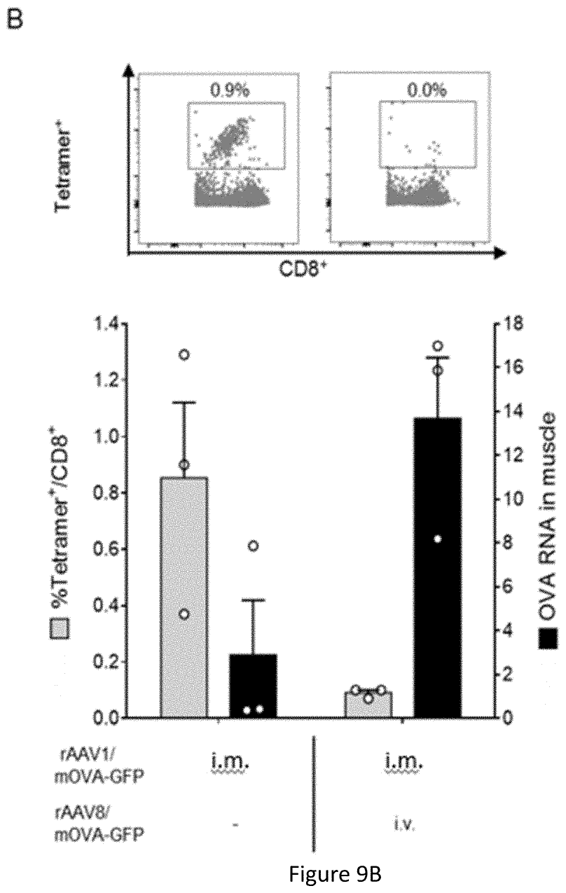

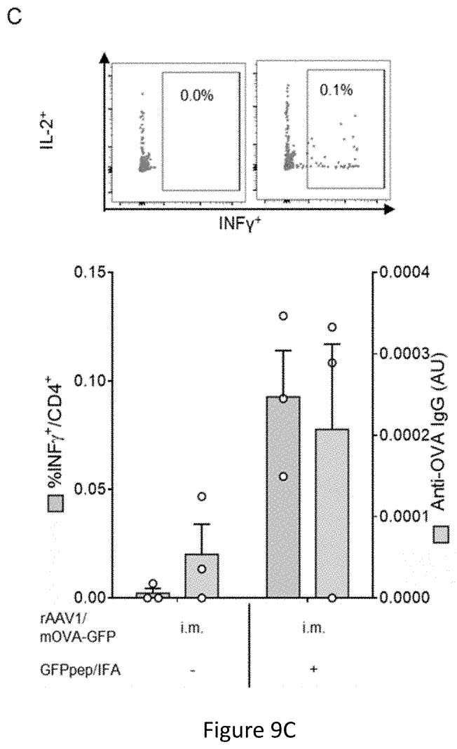

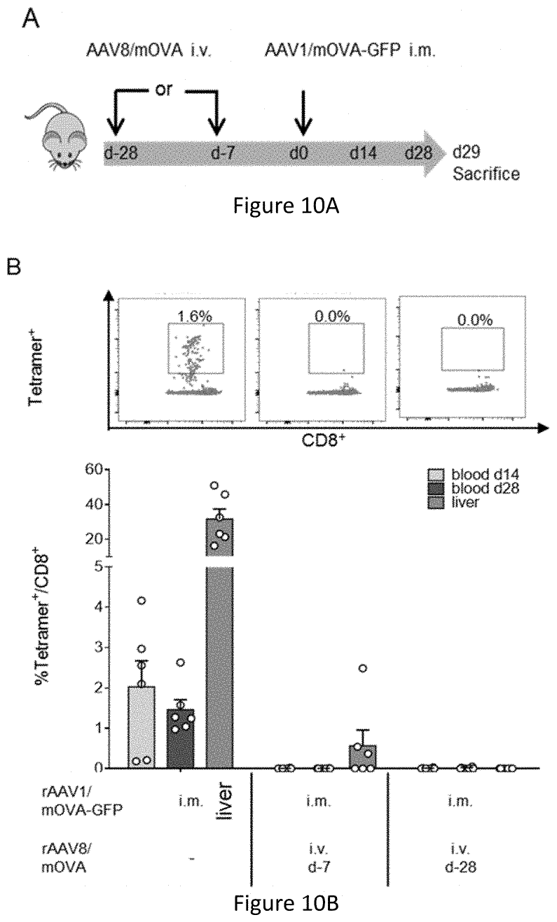

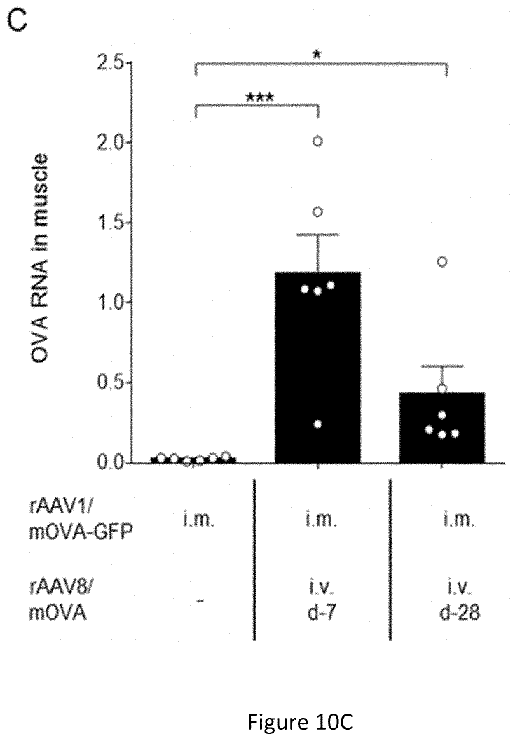

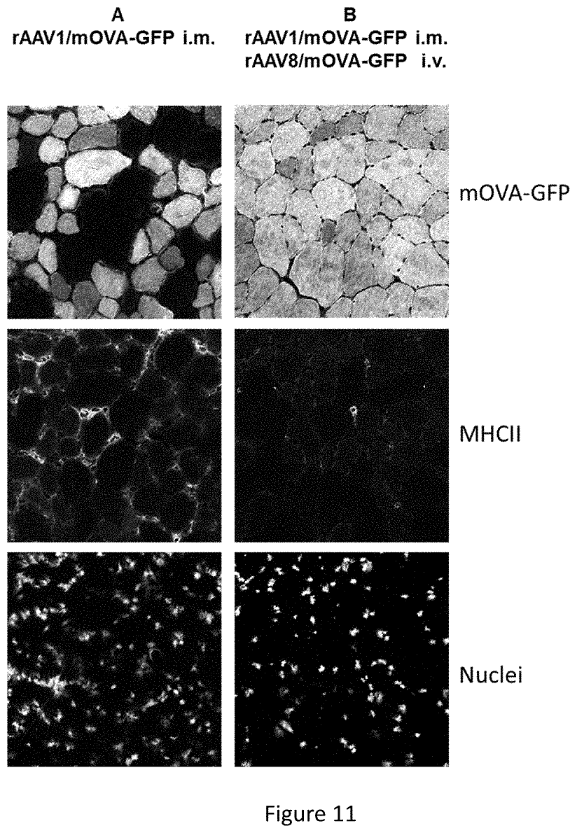

Male C57/BI6 mice were injected i.m. in the left tibialis anterior muscle with 10⁹ viral genomes (vg) of rAAV1 encoding mOVA under the muscle-specific Spc512 promotor and injected i.v. with 10¹⁰ vg of rAAV8 encoding hFIX under the liver-specific promotor hAAT. Lymphocytes were extracted from blood at day 14 and 28, and from liver at day 29 in order to analyze OVA-specific tetramer staining of CD8⁺T cells by cytometry. (A) Representative dot plots at d28 in blood. (B) Frequencies of CD8⁺ Kb/OVA257 tetramer⁺ (Tetramer⁺) in blood gated on CD8⁺T cells (C) Quantities of anti-OVA IgG relative to a control serum in arbitrary unit (AU). (D) RT-qPCR performed in muscle at day 29 in the experimental conditions listed. RT-qPCR results are expressed relatively to OVA RNA expression in the “rAAV1/mOVA i.m. & rAAV8/mOVA i.v.” group from FIG. 1. Each dot represents an individual animal, mean±SEM (n=9 mice per group, pooled from three independent experiments). ns p>0.05 (Mann-Whitney test)

FIG. 9. Tolerance Induction with Muscle and Liver Transduction of mOVA-GFP Transgene Bearing an Additional CD4 Epitope

Twelve male C57/BI6 mice were immunized or not with the MHCII GFP epitope at day 0 and injected at day 14 with 2,5×10¹⁰ vg of rAAV1 i.m. encoding mOVA/GFP under the muscle-specific Spc512 promotor and injected with 10¹⁰ vg of rAAV8 encoding for mOVA/GFP i.v. under the liver-specific promotor hAAT. Mice were euthanized at day 29 to collect spleen and injected muscle. Lymphocytes were extracted from spleen to perform Kb/OVA257 tetramer staining in CD8⁺ T cells and intracellular INFy staining gated on CD4⁺T cells. (A) Time line of the experiment. (B) Representative dot plots (upper panel) and frequencies of CD8⁺ Kb/OVA257 Tetramer⁺ (Tetramer⁺) in CD8⁺T cells (left axis) and expression of OVA RNA in muscle (right axis, lower panel) at d28 in the two experimental conditions listed (lower panel). (C) Representative dot plots of CD4+CD44⁺ INFy⁺ T cells (upper panel), frequencies of INFy⁺ gated on CD4+CD44^{hi}T cells in spleen (left axis) and amounts of blood anti-OVA IgG relative to a control serum in arbitrary unit (AU, right axis, lower panel) at d28 in the two experimental conditions listed (lowed panel). Each dot represents an individual animal, mean±SEM (n=3 mice per group).

FIG. 10. Induction of Sustained Immune Tolerance with rAAV8/mOVA Liver Delivery and Disparate mOVA-GFP Muscle Delivery

Male C57/BI6 mice were injected i.v. with 10¹⁰ vg of rAAV8/mOVA at day −28 or −7 or none (control group). At day 0, mice were injected in the left tibialis anterior muscle with 2,5×10¹⁰ vg of rAAV1/mOVA-GFP i.m. Blood was collected at d14 and d28 and mice euthanized at d29 to collect injected muscle and liver. (A) Time line of the experiment. (B) Representative dot plots in blood (upper panel) and frequencies of CD8⁺ Kb/OVA257 Tetramer⁺ (Tetramer⁺) gated on CD8⁺T cells (lower panel) at d28 in the three experimental conditions listed (lowed panel). (C) RT-qPCR to quantify OVA RNA expression in muscle at day 29 relative to the “rAAV1/mOVA-GFP i.m. & rAAV8/mOVA i.v. d-7” group. Each dot represents an individual animal, mean±SEM (n=6 mice per group, pooled from two independent experiments). **p<0.01 (Mann-Whitney test).

FIG. 11. Lack of Local Inflammation in Muscle Following Dual Muscle and Liver Transduction of mOVA-GFP Transgene.

Representative immunostaining images of muscle sections from mice injected in left tibialis anterior muscle with 10¹⁰ viral genomes (vg) of rAAV1 encoding mOVA-GFP under the muscle-specific SPc5-12 promotor and injected (B) or not (A) i.v. with 1×10¹⁰ vg rAAV8 encoding mOVA-GFP under the liver-specific promoter hAAT. mOVA-GFP: GFP labeling of muscle fibers expressing mOVA-GFP transgene; MCHII: MHCII labeling of the cells; DAPI: nuclei immunostaining.

### DETAILED DESCRIPTION OF THE INVENTION

The inventors used a highly immunogenic OVA transgene well adapted to decipher specific T cell responses and surprisingly found that dual muscle-liver rAAV transduction imposes sustained tolerance to both anti-transgene CD8⁺T cell and humoral responses, generated by rAAV muscle transduction. Importantly, in the presence of preimmune material comprising CD8⁺ and CD4⁺T cells, this tolerance induction is shown to operate through partial deletion of and induction of exhaustion in transgene specific CD8⁺T cells. Thus, the inventors found that liver rAAV transduction imposes immune tolerance to an entire transgene-specific T cell repertoire elicited after muscle transduction, regardless of pre-existing immune responses to the transgene.

To remedy and prevent immune reactions after the administration of a rAAV vector, the present invention provides a combination of two recombinant adeno-associated viral (rAAV) vectors comprising:

i. a first recombinant adeno-associated viral (rAAV) vector comprising a capsid and a cassette comprising a 5′ ITR sequence, a liver-specific promotor, a nucleic acid sequence of interest useful to be tolerated by the immune system, a poly A chain, wherein the nucleic acid sequences is administered to be delivered toward the liver, and

ii. a second recombinant adeno-associated viral (rAAV) vector comprising a capsid and a cassette comprising a 5′ ITR sequence, a promotor specific for a tissue of interest, a nucleic acid sequence corresponding to the nucleic acid sequence inserted into the cassette of the first rAAV vector, a poly A chain, wherein the nucleic acid sequence is administered to the tissue of interest,

for use as a drug in inducing an immune tolerance to a protein product encoded and translated from said nucleic acid sequence delivered to the said tissue of interest in a subject

### Definition

As intended herein, the term “comprising” has the meaning of “including” or “containing”, which means that when an object “comprises” one or several elements, other elements than those mentioned may also be included in the object. In contrast, when an object is said to “consist of” one or several elements, the object cannot include other elements than those mentioned.

According to the invention, the terms “subject”, “individual”, and “patient” are used interchangeably herein and refer to a mammal affected or likely to be affected with disease that can be treated with gene therapy. Subjects are preferably humans.

“Treating” or treatment of a disease or condition refers to any act intended to ameliorate the health status of patients. “Treatment” can include, but is not limited to, alleviation or amelioration of one or more symptoms or conditions, diminishment of extent of disease, stabilization of the state of disease (e.g. maintaining a patient in remission), prevention of the disease or prevention of the spread of disease, delay or slowing of disease progression, amelioration or palliation of the disease state, diminishment of the reoccurrence of disease, and remission (whether partial or total). A treatment may include curative, alleviation or prophylactic effects. The term “prophylactic” may be considered as reducing the severity or the onset of a particular condition. “Prophylactic” also includes preventing reoccurrence of a particular condition in a patient previously diagnosed with the condition. “Therapeutic” may also reduce or delay the severity of an existing condition. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition.

The term “combination”, “combinatorial treatment” or “combined therapy” when related to combination of the invention of a first and second rAAV vectors, designates a treatment wherein said first and second rAAV vectors are co-administered to a subject to cause a biological effect that is obtained because of the co-administration, e. g. immunotolerance and/or improve expression for a therapeutic peptide or protein. In a combined therapy according to this invention, said first and second rAAV vectors may be administered at the same time, together or separately, or sequentially. Also, they may be administered through different routes and protocols. For example, one of the vectors can be administered intravenously and the other intramuscularly. They may be administered through the same route and protocol, e.g. both intravenously. Also, first and second rAAV vectors might be formulated together, they might also be formulated separately.

As used herein, the terms “disorder” or “disease” refer to the incorrectly functioning organ, part, structure, or system of the body resulting from the effect of genetic or developmental errors, infection, poisons, nutritional deficiency or imbalance, toxicity, or unfavourable environmental factors. Preferably, these terms refer to a health disorder or disease e.g. an illness that disrupts normal physical or mental functions. More preferably, the term disorder refers to immune and/or inflammatory diseases that affect animals and/or humans.

The term “immune disease” or “auto-immune disease”, as used herein, refers to a condition in a subject characterized by cellular, tissue and/or organ injury caused by an immunologic reaction of the subject to its own cells, tissues and/or organs.

### The Recombinant Adeno-Associated Virus (rAAV)

“Recombinant Adeno-Associated Virus” or “rAAV” uses an exchange of nucleotide sequences to enable insertion, deletion or replacement of DNA sequences in cells. Unlike other gene editing methods, this is achieved without causing a double strand DNA break, instead stimulating endogenous homologous recombination. Due to its non-pathogenic nature, it is also suitable for gene therapy in live patients. In particular, the rAAV genome is built of single-stranded deoxyribonucleic acid (ssDNA), either positive- or negative-sensed, which is about 4.7 kilobase long. These single-stranded DNA viral vectors have high transduction rates and have a unique property of stimulating endogenous HR without causing double strand DNA breaks in the genome. The rAAV is particularly adapted to be use in gene therapy for example in ocular disease such as LUXTURNA™ (voretigene neparvovec-rzyl; Spark Therapeutics, Inc., Philadelphia, Pa.), who delivers a normal copy of the RPE65 gene to retinal cells for the treatment of biallelic RPE65 mutation-associated retinal dystrophy. Another one example of gene therapy using an rAAV vector and which has been approved in Europe in November 2012 is Alipogene tiparvovec (marketed under the trade name GLYBERA™). This gene therapy treatment is designed to reverse lipoprotein lipase deficiency (LPLD), a rare inherited disorder which can cause severe pancreatitis. The adeno-associated virus serotype 1 (AAV1) viral vector delivers an intact copy of the human lipoprotein lipase (LPL) gene to muscle cells. The injection is followed by immunosuppressive therapy to prevent immune reactions to the virus.

The present invention also relates to the use of a combination of two recombinant adeno-associated viral (rAAV) vectors comprising:

i. a first recombinant adeno-associated viral (rAAV) vector comprising a capsid and a cassette comprising a 5′ ITR sequence, a liver-specific promotor, a nucleic acid sequence of interest useful to be tolerated by the immune system, a poly A chain, wherein the nucleic acid sequences is administered to be deliver toward the liver, and

ii. a second recombinant adeno-associated viral (rAAV) vector comprising a capsid and a cassette comprising a 5′ ITR sequence, a promotor specific for a tissue of interest, a nucleic acid sequence corresponding to the nucleic acid sequence inserted into the cassette of the first rAAV vector, a transmembrane sequence, a poly A chain, wherein the nucleic acid sequences is administered towards to the tissue of interest,

for use as a drug in inducing an immune tolerance to a protein product encoded and translated from said nucleic acid sequence delivered to the said tissue of interest in a subject.

A particular embodiment of the invention relates to a combination of:

i. a first recombinant adeno-associated viral (rAAV) vector comprising a capsid and a cassette comprising a 5′ ITR sequence, a liver-specific promotor, a nucleic acid sequence coding for a protein product to be tolerated by the immune system, a poly A chain, and

ii. a second recombinant adeno-associated viral (rAAV) vector comprising a capsid and a cassette comprising a 5′ ITR sequence, a muscle-specific promotor, a nucleic acid sequence coding for the protein product to be tolerated by the immune system of the first recombinant adeno-associated viral vector, a poly A chain,

for use in inducing an immune tolerance for the said protein product encoded by either first or second r AAV vector, wherein the first rAAV is administered to target the liver and the second rAAV is administered to target muscle tissues.

Another particular embodiment of the invention relates to a combination of:

i. a first recombinant adeno-associated viral (rAAV) vector comprising a capsid and a cassette comprising a 5′ ITR sequence, a liver-specific promotor, a nucleic acid sequence coding for a protein product to be tolerated by the immune system, a poly A chain, and

ii. a second recombinant adeno-associated viral (rAAV) vector comprising a capsid and a cassette comprising a 5′ ITR sequence, a muscle-specific promotor, a nucleic acid sequence coding for the protein product to be tolerated by the immune system of the first recombinant adeno-associated viral vector a transmembrane sequence, a poly A chain,

for use in inducing an immune tolerance for the said protein product encoded by either first or second rAAV vector, wherein the first rAAV is administered to target the liver and the second rAAV is administered to target muscle tissues.

In a particular embodiment, the cassette inserted in the recombinant adeno-associated viral (rAAV) vector comprises a nucleic acid sequence coding for a cell-associated protein product. By “cell-associated protein product”, the invention refers to a specific sequence allowing either to anchor the protein to the membrane via a transmembrane domain or by default, to deliver the protein to the cytosol in the absence of a leader sequence. Preferably, the nucleic acid sequence of interest inserted in the cassette is coding for a transmembrane protein product.

In a preferred embodiment, the present invention relates to a combination of two recombinant adeno-associated viral (rAAV) vectors, wherein the nucleic acid sequence of interest inserted in the cassette is coding for a protein product comprising an epitope recognized by T-cells or B-cells. By “nucleic acid sequence” or “transgene”, the invention refers to a sequence of nucleic acid considered of interest with a therapeutically action for the treatment of a disease. Preferably the nucleic acid sequence of interest is therapeutically effective. Preferably, the transgene encodes a therapeutic (poly)peptide or therapeutic protein and refers herein to “therapeutic protein product”, “protein product” or “protein product to be tolerated by the immune system”. Said protein product is notably effective in curing the defective activity in the cells by the replacement or by compensating the activity of the defective protein in a subject

In a particular embodiment, the cassette inserted in the first rAAV vector comprises a nucleic acid sequence coding for only an immunogenic part of the therapeutic protein, that is the part that contains the epitope recognized by either T- or B- cells and is responsible for adverse immune reaction, e.g. rejection of the transgene and/or low expression of the therapeutic protein product. In that case, the protein product that is expressed by the first rAAV vector might not exhibit the biological activity of the therapeutic protein either in its whole length or active part by replacing or compensating the defective activity of the cells, but only an activity toward the immune system of the subject.

In another particular embodiment, the cassette inserted in the first rAAV vector comprises a nucleic acid sequence coding for a protein product which is therapeutically effective in treating a disease by replacing or compensating the defective activity of the cells, even more particularly the protein product corresponds to the active protein in its whole length as described or known in the art.

In an even more particular embodiment, the protein product encoded by the first rAAV vector is only the immunogenic part of the therapeutic protein, that is the part that contains the epitope recognized by either T- or B- cells and is responsible for adverse immune reaction, whereas the protein product expressed by the second rAAV vector corresponds to a protein product that allows curing the disease by replacing or compensating the activity of the defective protein in a subject. Said protein product expressed by the second rAAV can be the active protein in its whole length as it is known in the art.

In another particular embodiment, the cassette of the second rAAV vector comprises a transmembrane nucleotide sequence to be fused to the sequence encoding the protein product so that, when expressed in the transduced cells, the protein product is maintained at the surface of the transduced cells through a transmembrane domain.

Therapeutic (poly)peptide and proteins for use in the context of the present invention include, but are not limited to, microdystrophin that represents an artificial form of dystrophin, Emerin, Lamin A/C, Spectrin repeat containing, nuclear envelope 1 (nesprin 1), Spectrin repeat containing, nuclear envelope 2 (nesprin 2), Transmembrane protein 43, Torsin A interacting protein 1, Double homeobox 4, Structural maintenance of chromosomes flexible hinge domain containing 1, Polymerase I and transcript release factor(M), Myotilin, Caveolin 3, HSP-40 homologue, subfamily B, number 6, Desmin, Transportin 3, Heterogeneous nuclear ribonucleoprotein D-like, Calpain 3, Dysferlin, Gamma sarcoglycan, Alpha sarcoglycan, Beta sarcoglycan, Delta-sarcoglycan, Telethonin, Tripartite motif-containing 32, Fukutin-related protein, Titin, Protein-O-mannosyltransferase 1, Anoctamin 5, Protein-O-mannosyltransferase 2, O-linked mannose beta1,2-N-acetylglucosaminyltransferase, Dystroglycan1, plectin, Desmin, trafficking protein particle complex 11, GDP-mannose pyrophosphorylase B, Isoprenoid synthase domain containing, Acid alpha-glucosidase preproprotein, LIM and senescent cell antigen-like domains 2, blood vessel epicardial substance, Torsin A interacting protein 1, Protein 0-Glucosyltransferase 1, Dolichyl-phosphate mannosyltransferase polypeptide 3, Valosin-containing protein, plectin.

The combination of the invention is particularly effective in eliminating or attenuating the occurrence of cellular and humoral immune responses to the protein product for which immune tolerance is sought. More particularly the combination allows a CD8⁺T cell immune tolerance and this despite preexisting humoral and CD8⁺T cell immunity toward the protein product encoded by the transgene.

Accordingly, in a particular embodiment, the invention relates to a combination of a first rAAV and a second rAAV vectors as exposed above, for its use in inducing immunotolerance toward the protein product encoded by the cassette of the rAAV vectors in a subject, wherein inducing immunotolerance comprises eliminating or attenuating the occurrence of cellular and humoral immune responses to the protein product for which immune tolerance is sought, e.g. therapeutic protein product. In another particular embodiment, the invention relates to a combination of a first rAAV and a second rAAV vectors as exposed above, for its use in inducing immunotolerance toward the protein product encoded by the cassette of the rAAV vectors in a subject, wherein inducing immunotolerance comprises allowing CD8⁺T cell immune tolerance. In another particular embodiment, the invention relates to a combination of a first rAAV and a second rAAV vectors as exposed above, for its use in inducing immunotolerance toward the protein product encoded by the cassette of the rAAV vectors in a subject, wherein the subject is with preexisting immunity toward said protein product.

When administered sequentially, the time interval between the administration of the first and second rAAV vector in the combination according to the present invention should be such that immune tolerance for the protein product encoded by the transgene should be at least maintained or even optimal.

The inventors have found that immune tolerance long after initial liver administration of the transgene for which immune tolerance is sought. Accordingly, in the combinations of the invention, the first rAAV vector is administered before the second rAAV vector, preferably one week, even more preferably one month before the second rAAV vector.

Advantageously, the nucleic acid sequence or transgene is coding for a protein product comprising an epitope recognized by T cells or B-cells. This epitope is recognized by the subject and can initiate a specific immune response, which is deleterious for the gene transfer operation and restauration of therapeutic expression level of the transgene coded by the said nucleic acid sequence.

In the context of the invention, the inventors surprisingly found that the combination of recombinant adeno-associated viral (rAAV) vectors can be administered to subjects who had a state of prior immunization of CD4, CD8 and/or B Lymphocytes. This aspect of the invention is particularly suited in clinical situations where preexisting immunity to the transgene is encountered. Patients that have spontaneous partial restauration of expression of the defective protein due to either rare exon skipping events, reversed gene mutations or that developed preexisting immunity to previously administered protein replacement therapy can develop such preexisting immunity to the transgene. In a more preferred embodiment, the combination is administered to the subject exhibiting a noticeable level of immunization of CD4, CD8 and/or B lymphocytes directed toward the protein product encoded by said nucleic acid sequence. According to the invention, the combination can be administered wherein the subject had a prior immunization status towards the protein product encoded and translated from said nucleic acid sequence. Preferably, the combination can be administered in a subject wherein the prior immunization status towards the protein product encoded and translated from said nucleic acid sequence yields the presence of CD4, CD8 and/or B Lymphocytes specific to the said protein product.

In a preferred embodiment, the combination of recombinant adeno-associated viral (rAAV) vectors comprising the cassette further comprises a leader peptide. The leader peptide could be only inserted in the first rAAV or in the second rAAV or both rAAV. In the context of the invention leader peptide is a short peptide particularly suited to be translated from bacterial leader RNA sequences which are involved in transcriptional or translation attenuation, mechanisms that modulate mRNA transcription or translation.

In a preferred embodiment, the combination of recombinant adeno-associated viral (rAAV) vectors according to the invention comprises in the first rAAV vectors a liver-specific promotor which is a hepatocyte-specific promotor (hAAT). The hepatocyte-specific promotor (hAAT) is particularly adapted to target the liver and more particularly the hepatocyte in gene therapy.

In another embodiment of the invention, the first and/or second recombinant adeno-associated viral (rAAV) vector comprise a capsid which is independently selected from the group consisting in AAV1, AAV2, AAV3, AAV4, AAV5, AAV6, AAV7, AAV8, AAV9, AAV10, AAV11 and any combination thereof. The capsid selected in the group above could be the same for the first and second rAAV vectors of the combination or the capsid of each first second rAAV vector of the combination of the invention is different. For example, and without limiting the scope, the first rAAV vector comprises a AAV7, a AAV8 or AAV9 capsid and the second rAAV vector comprises a AAV1, AAV7, AAV8 or AAV9 or AAV2 capsid. In another example, the first rAAV vector comprises a AAV8 or AAV9 capsid and the second rAAV vector comprises a AAV1 or AAV9 or AAV2 capsid.

The capsid of the adeno-associated viruses forms an icosahedron about 25 nm in diameter. It is constituted of structural proteins VP1 (viral protein 1), VP2 and VP3, assembled according to a ratio of 1:1:10. Tropism of the VAA variants is mainly determined by the loop domains of VP proteins. The mechanisms of entry of adeno-associated viruses into the target cells differ depending on the serotype. In general, adeno-associated virus infection begins with adherence to cellular receptors followed by internalization by endocytosis through secondary receptors. Following its endosomal and cytoplasmic transport, the virus is then decapsited and releases its DNA inside the nucleus.

Today, eleven serotypes capsid of AAV have been identified, with the best characterized and most commonly used being AAV2. These serotypes differ in their tropism, or the types of cells they infect, making AAV a very useful system for preferentially transducing specific cell types. The table below gives a summary of the tropism of AAV serotypes, indicating the optimal serotype(s) for transduction of a given organ.

TABLE 1
serotypes capsid of AAV and their optimal tropism.

| Tissue | Optimal Serotype |
|---|---|
| CNS | AAV1, AAV2, AAV4, |
| | AAV5, AAV8, AAV9 |
| Heart | AAV1, AAV8, AAV9 |
| Kidney | AAV2 |
| Liver | AAV7, AAV8, AAV9 |
| Lung | AAV4, AAV5, AAV6, AAV9 |
| Pancreas | AAV8 |
| Photoreceptor Cells | AAV2, AAV5, AAV8 |
| RPE (Retinal | AAV1, AAV2, AAV4, |
| Pigment | AAV5, AAV8 |
| Epithelium) | |
| Skeletal Muscle | AAV1, AAV6, AAV7, |
| | AAV8, AAV9 |

In the context of the invention, the capsid is preferably selected from the group consisting in a AAV1, a AAV6, a AAV7, a AAV8 and a AAV9 capsid. When contemplating inducing immune tolerance in the context of treating a muscular dystrophy, the capsid is more preferably selected from the group consisting in a AAV7, a AAV8, and a AAV9, even more preferably the capsid is a AAV8 capsid.

As shown in the experimental section, in the muscle, elimination or attenuation of cellular and humoral immune responses to the protein product afforded by the combination according to the invention results in nullifying local inflammation in muscle tissue that is otherwise observed when no immunotolerance is induced. Further, an improvement in the expression of the protein product in transduced cells is observed. These features made combination of the invention of particular interest for the treatment of muscular dystrophies.

Accordingly, a particular embodiment of the invention relates to a combination of:

i. a first recombinant adeno-associated viral (rAAV) vector comprising a capsid and a cassette comprising a 5′ ITR sequence, a liver-specific promotor, a nucleic acid sequence coding for a protein product to be tolerated by the immune system, a poly A chain, and

ii. a second recombinant adeno-associated viral (rAAV) vector comprising a capsid and a cassette comprising a 5′ ITR sequence, a muscle-specific promotor, a nucleic acid sequence coding for the protein product to be tolerated by the immune system of the first recombinant adeno-associated viral vector, a poly A chain,

for use in treating a muscular dystrophy, preferably a monogenic muscle disorder, in a subject, wherein the first rAAV is administered to target the liver and the second rAAV is administered to target muscle tissues.

Also, a more particular embodiment of the invention relates to a combination of:

i. a first recombinant adeno-associated viral (rAAV) vector comprising a capsid and a cassette comprising a 5′ ITR sequence, a liver-specific promotor, a nucleic acid sequence coding for a protein product to be tolerated by the immune system, a poly A chain, and

ii. a second recombinant adeno-associated viral (rAAV) vector comprising a capsid and a cassette comprising a 5′ ITR sequence, a muscle-specific promotor, a nucleic acid sequence coding for the protein product to be tolerated by the immune system of the first recombinant adeno-associated viral vector, a transmembrane sequence, a poly A chain,

for use in treating a muscular dystrophy, preferably a monogenic muscle disorder, in a subject, wherein the first rAAV is administered to target the liver and the second rAAV is administered to target muscle tissues.

Accordingly, in a particular embodiment, the invention relates to a combination of a first rAAV and a second rAAV vectors as exposed above, for its use in treating a muscular dystrophy, wherein treating a muscular dystrophy comprises eliminating or attenuating the occurrence of cellular and humoral immune responses to the protein product for which immune tolerance is sought, allowing said protein product to be tolerated by the immune system and/or its expression in muscle of the subject. In another particular embodiment, treating a muscular dystrophy comprises allowing CD8⁺T cell immune tolerance toward the protein product in the subject. In a more preferred embodiment, the invention relates to a combination of the first recombinant adeno-associated viral (rAAV) vector as described above and a second recombinant adeno-associated viral (rAAV) vector, wherein the nucleic acid sequence of interest is coding for a muscle associated protein, preferably a membrane protein.

More particularly, the second recombinant adeno-associated viral (rAAV) vector comprising a capsid and a cassette comprising a 5′ ITR sequence, a muscle-specific promotor, a nucleic acid sequence corresponding to the nucleic acid sequence inserted into the cassette of the first rAAV vector, a transmembrane sequence, a poly A chain, wherein the nucleic acid sequences is administered to the muscle, for use as a drug in inducing an immune tolerance to a protein product encoded and translated from said nucleic acid sequence in a subject.

In another embodiment, the nucleic acid sequence of interest is coding for a muscle specific protein or a neuromuscular protein.

In a particular embodiment of the invention, the combination comprises in the two rAAV vectors a nucleic acid sequence coding for the sequence selected in the group consisting in microdystrophin constructs, Emerin, Lamin A/C, Spectrin repeat containing, nuclear envelope 1 (nesprin 1), Spectrin repeat containing, nuclear envelope 2 (nesprin 2), Transmembrane protein 43, Torsin A interacting protein 1, Double homeobox 4, Structural maintenance of chromosomes flexible hinge domain containing 1, Polymerase I and transcript release factor(M), Myotilin, Caveolin 3, HSP-40 homologue, subfamily B, number 6, Desmin, Transportin 3, Heterogeneous nuclear ribonucleoprotein D-like, Calpain 3, Dysferlin, Gamma sarcoglycan, Alpha sarcoglycan, Beta sarcoglycan, Delta-sarcoglycan, Telethonin, Tripartite motif-containing 32, Fukutin-related protein, Titin, Protein-O-mannosyltransferase 1, Anoctamin 5, Protein-O-mannosyltransferase 2, O-linked mannose beta1,2-N-acetylglucosaminyltransferase, Dystroglycan1, plectin, Desmin, trafficking protein particle complex 11, GDP-mannose pyrophosphorylase B, Isoprenoid synthase domain containing, Acid alpha-glucosidase preproprotein, LIM and senescent cell antigen-like domains 2, blood vessel epicardial substance, Torsin A interacting protein 1, Protein 0-Glucosyltransferase 1, Dolichyl-phosphate mannosyltransferase polypeptide 3, Valosin-containing protein, plectin.

More preferably, the combination of the first recombinant adeno-associated viral (rAAV) vector and the second recombinant adeno-associated viral (rAAV) vector are used for the treatment of Duchenne Muscular Dystrophy (DMD).

According to an alternative embodiment, the present invention relates to a recombinant adeno-associated viral (rAAV) vector comprising a capsid and a cassette comprising a 5′ ITR sequence, a liver-specific promotor and a second promotor specific for a tissue of interest, a nucleic acid sequence of interest useful to be tolerated by the immune system, a poly A chain, wherein the nucleic acid sequences is administered to be deliver toward the liver and toward to the tissue of interest for use as a drug in inducing an immune tolerance to a protein product encoded and translated from said nucleic acid sequence delivered to the said tissue of interest in a subject.

### Pharmaceutical Composition

In another aspect, the invention relates to a pharmaceutical composition comprising a first recombinant adeno-associated viral (rAAV) vector and a second recombinant adeno-associated viral (rAAV) vector comprising a capsid and a cassette comprising a 5′ ITR sequence, a promotor specific for a tissue of interest, a nucleic acid sequence corresponding to the nucleic acid sequence inserted into the cassette of the first rAAV vector, a transmembrane sequence, a poly A chain, wherein the nucleic acid sequences is administered towards to the tissue of interest, for use as a drug in inducing an immune tolerance to said nucleic acid sequence delivered to the said tissue of interest in a subject.

The first rAAV described above is a rAAV comprising a capsid and a cassette comprising a 5′ ITR sequence, a liver-specific promotor, a nucleic acid sequence of interest useful to be tolerated by the immune system, a poly A chain, wherein the nucleic acid sequences is administered to be delivered to the liver. The nucleic acid sequence of interest useful to be tolerated by the immune system corresponds to the transgene of interest useful as a drug and is therapeutically effective. By transgene of interest, the invention refers to any product of said transgene. In the context of the invention the product of the transgene of interest is useful as a drug and is therapeutically effective too.

The pharmaceutical composition of the invention relates to a first rAAV delivered to the liver comprising a transgene of interest which is therapeutically active and useful for the treatment of a disease in a subject and a second rAAV comprising a transgene corresponding to the nucleic acid sequence inserted into the cassette of the first rAAV vector and is useful to be delivered to the tissue of interest, for example the muscle.

In a preferred embodiment, the first and the second recombinant adeno-associated viral (rAAV) is administered simultaneously or sequentially, preferably simultaneously.

In a particular aspect of the invention, the pharmaceutical composition could be administered two or three times, as many times as desired and/or repetitively. In a preferred embodiment the second recombinant adeno-associated viral (rAAV) administration is followed by a third adeno-associated viral (rAAV) administration using a rAAV with a different serotype comprising a transgene corresponding to the nucleic acid sequence inserted into the cassette of the first rAAV vector and is useful to be delivered to the tissue of interest, for example the muscle. This process could be repeated several times with rAAV bearing non cross reactive serotypes.

In a preferred embodiment of the invention the pharmaceutical composition is administered intravenously or intramuscularly. Of course, it is possible to administer the composition according to other modes of administration depending on the diseases, for example intraocular injection.

According to the invention, the pharmaceutical composition is particularly suited to be used in gene therapy, preferably a gene therapy of monogenic disorders. Multiple gene therapy treatments could be feasible, but the pharmaceutical composition according to invention is particularly adapted to be used in muscular dystrophies, preferably a monogenic muscle disorder, more preferably the Duchenne Muscular Dystrophy. In another embodiment, the pharmaceutical composition is useful in treating auto-immune disorders using rAAV vector comprising a transgene coding for the protein targeted by the autoreactive B and T lymphocytes.

### Method

In another aspect, the invention relates to a method for preventing the rejection of a transgene of interest or for protecting a transgene of interest comprising the step of administrated a first rAAV according to the invention and described in the experimental part below, wherein the nucleic acid of interest into the rAAV is the nucleic acid corresponding to the transgene of interest.

In an embodiment, a method according to the invention relates to a method for inducing an immune tolerance to a protein product in a subject, said method comprising the steps of:
administering to the subject a first recombinant adeno-associated viral (rAAV) vector comprising a capsid and a cassette comprising a 5′ ITR sequence, a liver-specific promotor, a nucleic acid sequence coding for a protein product to be tolerated by the immune system, a poly A chain, and
administering to the subject a second recombinant adeno-associated viral (rAAV) vector comprising a capsid and a cassette comprising a 5′ ITR sequence, a muscle-specific promotor, a nucleic acid sequence coding for the protein product to be tolerated by the immune system of the first recombinant adeno-associated viral vector a transmembrane sequence, a poly A chain,
   thereby allowing the prevention, or the lowering, of the rejection of the protein product to be tolerated by the immune system encoded by the first and/or second rAAV vector. Said method is of particular interest in treating a muscular dystrophy, preferably a monogenic muscle disorder, even more preferably Duchenne Muscular Dystrophy (DMD).

In one embodiment, in the method of the invention, each of rAAV vectors can be administered repeatedly to the subject, in order to improve immune tolerance and/or expression of the protein to be tolerated, encoded by the transgene of interest.

Also, in a particular embodiment, the method comprises the repeated administration to the subject of a first rAAV as described above, in order to obtain the desired immune tolerance and/or expression of the protein to be tolerated. In even more particular embodiment, the rAAV is administrated once, twice or even three times to the subject.

In another particular embodiment, the method comprises the repeated administration to the subject of the second rAAV as described above, in order to obtain the desired immune tolerance and/or expression of the protein to be tolerated. In even more particular embodiment, the rAAV is administrated once, twice or even three times to the subject.

In another particular embodiment, the administration of a first and second rAAV as described above is separated in time, in order, for example, to obtain the desired immune tolerance induction in liver at the time of the administration of a second rAAV, thereby optimizing the effect of the administration of the second rAAV on immune tolerance toward the protein encoded by the transgene for which induction of immune tolerance is sought. Surprisingly, the method is particularly adapted to be administered after a prior immunization with said transgene of interest.

In a particular embodiment, the method of the invention further comprises a step of testing or detecting the presence of a preexisting immunity toward the protein product to be tolerated. Said detection can be performed easily by using any immunological method well known by the skilled in the art, for example, for detecting the presence, in the subject, of antibodies, or reactive immune cells, to the protein encoded by the transgene for which immune tolerance is sought. This step can be performed either before the administration steps of the two rAAV vectors as described above. This can be of interest in order to adapt the treatment to the subject, e.g., in determining the number of administrations of each of rAAV vector or the duration of the treatment. This step can be also applied after the administration steps of the two rAAV vectors as described above, to, e.g., evaluate the induction of the of immune tolerance in the subject.

### Kits for Therapeutic Methods and Uses of the Invention

The invention also relates to a kit suitable to implement any of therapeutic use, method or composition of the invention.

Accordingly, a further object of the invention is a kit comprising:
a first recombinant adeno-associated viral (rAAV) vector comprising a capsid and a cassette comprising a 5′ ITR sequence, a liver-specific promotor, a nucleic acid sequence coding for a protein product to be tolerated by the immune system, a poly A chain, and
a second recombinant adeno-associated viral (rAAV) vector comprising a capsid and a cassette comprising a 5′ ITR sequence, a muscle-specific promotor, a nucleic acid sequence coding for the protein product to be tolerated by the immune system of the first recombinant adeno-associated viral vector a poly A chain.

In a particular embodiment said kit comprises:
a first recombinant adeno-associated viral (rAAV) vector comprising a capsid and a cassette comprising a 5′ ITR sequence, a liver-specific promotor, a nucleic acid sequence coding for a protein product to be tolerated by the immune system, a poly A chain, and
a second recombinant adeno-associated viral (rAAV) vector comprising a capsid and a cassette comprising a 5′ ITR sequence, a muscle-specific promotor, a nucleic acid sequence coding for the protein product to be tolerated by the immune system of the first recombinant adeno-associated viral vector a transmembrane sequence, a poly A chain.

In another particular embodiment, said kit further comprises instructions for the use of each of the said first and second rAAV in inducing an immune tolerance to the protein product to be tolerated and which is encoded and translated from nucleic acid sequence delivered by each of said first and second rAAV.

In a more particular embodiment said kit further comprises instructions for the use of each of the said first and second rAAV in inducing an immune tolerance to the protein product to be tolerated and which is encoded and translated from nucleic acid sequence delivered by each of said first and second rAAV, for treating a muscular dystrophy, preferably a monogenic muscle disorder, in a subject. More particularly, in a further embodiment the subject is suffering from the Duchenne Muscular Dystrophy (DMD). In this regard, in a kit according to the invention, said first rAAV and second rAAV comprise anyone of the features as described above for the rAAV vectors, therapeutics methods or uses and pharmaceutical composition of the invention.

As exposed above, in the therapeutic uses or methods according to the invention, the first and second rAAV can be administered to the subject in the same formulation or separately, in a more or less long time interval. Also the invention is also related to a kit suitable to the transduction of the liver and another kit suitable to the transduction of the muscle, in the frame of the use or methods of the invention.

In a more further embodiment, the invention relates to a kit comprising:
a recombinant adeno-associated viral (rAAV) vector comprising a capsid and a cassette comprising a 5′ ITR sequence, a liver-specific promotor, a nucleic acid sequence coding for a protein product to be tolerated by the immune system, a poly A chain, and
instructions for the use of said rAAV in inducing an immune tolerance to the protein product to be tolerated and which is encoded and translated from nucleic acid sequence delivered by said rAAV, more particularly for targeting and transduction in the liver of the subject, even more particularly for treating a muscular dystrophy, preferably a monogenic muscle disorder, in a subject.

In another further embodiment, the invention relates to a kit comprising:
a recombinant adeno-associated viral (rAAV) vector comprising a capsid and a cassette comprising a 5′ ITR sequence, a muscle-specific promotor, a nucleic acid sequence coding for the protein product to be tolerated by the immune system, a poly A chain, and
instructions for the use of said rAAV in inducing an immune tolerance to the protein product to be tolerated and which is encoded and translated from nucleic acid sequence delivered by said rAAV, more particularly for targeting and transduction in the skeletal muscles of the subject, even more particularly for treating a muscular dystrophy, preferably a monogenic muscle disorder, in a subject.

In another further embodiment, the invention relates to a kit comprising:
a recombinant adeno-associated viral (rAAV) vector comprising a capsid and a cassette comprising a 5′ ITR sequence, a muscle-specific promotor, a nucleic acid sequence coding for the protein product to be tolerated by the immune system, a transmembrane sequence, a poly A chain, and
instructions for the use of said rAAV in inducing an immune tolerance to the protein product to be tolerated and which is encoded and translated from nucleic acid sequence delivered by said rAAV, more particularly for targeting and transduction in the skeletal muscles of the subject, even more particularly for treating a muscular dystrophy, preferably a monogenic muscle disorder, in a subject.

The use of kits dedicated to either liver or muscle targeting is of particular interest where the rAAV comprising a liver specific promoter and the rAAV comprising the muscle specific promoter of the combination of the invention are to be administered sequentially to the subject, even more when administered through different routes. A further interest for the two separates kits lies in cases where a time interval is applied between the targeting of the liver and the targeting of the muscle, even more when repeated administrations of the first AAV are applied, e. g. to obtain the desirable liver specific immune tolerance, before administration of the second rAAV vector targeting the muscle. Also, repeated administrations with the rAAV with the muscle specific promoter can be applied, e. g. to obtain the desirable immune tolerance and or expression by muscle cells of the protein product to be tolerated.

In the kits according to the invention, each of the rAAV vector with either the liver or muscle specific promoter is in unit dosage form depending on, e.g., the route of administration or the number of administrations of each of said rAAV vectors. Advantageously, said kits can comprise the number of unit dosage forms suitable to obtain the desirable immune tolerance for the peptide to be tolerated.

In particular embodiment, a kit according to the invention further comprises a mean for testing or detecting a preexisting immunity toward the protein product encoded in the rAAV vectors, in order for example to determine the therapeutic protocol to induce a proper immunotolerance and/or an effective treatment, e.g. by adapting the doses or number of administration of each of the first and second rAAV vectors.

Further aspects and advantages of the invention will be disclosed in the following examples, which should be considered illustrative.

### EXAMPLE

### Material & Methods

### Mice and In Vivo Injections

6- to 8-week-old C57BL/6JRj male mice were purchased from JANVIER LABS, housed under specific pathogen-free conditions in the animal facility, and handled in accordance with French and European directives. For intramuscular injection, mice were anesthetized using isofluran and indicated doses of rAAV vector, diluted in 25 μL PBS, were injected into the left tibialis anterior using a 30 G RN Hamilton syringe. For intravenous injection 200 μL of the indicated rAAV vector diluted in PBS was injected in the caudal vein using a 0.5 mL insulin Myjector U-100 syringe (TERUMO).

### Plasmid Constructions and Recombinant AAV Vector Productions

mOVA19 cDNA were inserted by PCR in pSMD2 rAAV1 or rAAV8 plasmid between the SPc5-12 muscle-specific promoter or hAAT hepatocyte-specific promotor and a polyA signal to create rAAV1/SPc5-12-mOVA (rAAV1/mOVA) targeting the muscle or rAAV8/hAAT-mOVA (rAAV8/mOVA) targeting the liver, respectively. Alternatively, full length cDNA sequence for the GFP protein was fused to the transmembrane domain of the mOVA cDNA, to create mOVA-GFP sequence and as described above, rAAV1/SPc5-12-mOVA-GFP (rAAV1/mOVA-GFP) and rAAV8/hAAT-mOVA-GFP (rAAV8/mOVA-GFP). All AAV vectors used in this study were produced using an adenovirus-free transient transfection method and purified as described earlier (Vidal et al., 2018). Titers of the AAV vector stocks were determined using a real-time qPCR and confirmed by SDS-PAGE, followed by SYPRO Ruby protein gel stain and band densitometry.

Quantification of OVA mRNA

Total RNA were extracted from twenty 12 μm frozen sections of each organ using the Nucleospin RNA plus kit (MACHEREY-NALGEL, Düren, Germany). For quantification of OVA mRNA, 100 ng of total RNA were reverse transcribed using Superscript II kit (Invitrogen). Then 4 μL of RT-PCR product were subjected to real-time PCR amplification using Ova-F (5′-AAGCAGGCAGAGAGGTGGTA-3′) (SEQ ID NO: 1), Ova-R (5′-GAATGGATGGTCAGCCCTAA-3′) (SEQ ID NO: 2), β-actin-F (5′-AAGATCTGGCACCACACCTTCT-3′) (SEQ ID NO: 3), and β-actin-R (5′-TTTTCACGGTTGGCCTTAGG-3′) (SEQ ID NO: 4) primers. All reaction mixtures were made according to QuantiFAst SYBR Green PCR kit instruction (QUIAGEN, Germany), OVA primers were used at 500 nmol/I and β-actin primers at 400 nmol/I, as described previously. The absolute amount of OVA mRNA for each sample was calculated and normalized using the ΔΔCt formula: 1/(2\hat{ }(−(Ctβactin-CtOVA)sample-(Ctβactin-CtOVA) reference). The reference used in this formula is the mean ΔCt value of the double injected “rAAV1/mOVA i.m. and rAAV8/mOVA iv” group defined in each experiment.

### Lymphocytes Isolation

For peripheral blood lymphocyte isolation, erythrocytes were eliminated by hypotonic shock with BD Pharm Lyse buffer (BD Biosciences). For splenocytes isolation, spleen was crushed manually in 1×PBS 0.1% HSA. For isolation of lymphocytes from liver, liver was collected and crushed manually in 1×PBS 0.1% HSA, then resuspended in 4 mL of 1×PBS 0.1% HSA and spun at 30 g for 2 minutes at 4° C. in order to eliminate cellular debris. Supernatant was spun at 300 g for 5 minutes at 4° C. The cell pellet was resuspended in 40% Percoll (Sigma, USA) at room temperature. 2 mL of 70% Percoll solution at room temperature was then added below the 40% cell suspension. Percoll gradient was centrifuged at 1300 g for 20 minutes at room temperature with no break. The upper fat layer was removed, and the interface cell band was collected.

### Flow Cytometry Analysis

For tetramer staining, we followed the method previously described by Ghenassia A et al. (2017) or Gross DA et al. (2019). Briefly, cell suspensions were first incubated with iTAg Tetramer/PE—H-2 Kb OVA (Clinisciences, Nanterre, France) for 30 minutes at room temperature, then blocked with anti-CD16/CD32 antibody (2.4 G2, Bio X Cell) for 10 minutes at 4° C. followed by membrane staining for 15 min at 4° C. using a combination of BV421 or APC anti-CD8α (53-6.7), fluorescein isothiocyanate (FITC) anti-CD44 (IM7), BV421 anti-PD-1 (29F.1A12), PE-Cy7 anti-CD4 (RM4-5).

For intracellular staining, cell suspensions were first blocked with anti-CD16/CD32 antibody (2.4G2, Bio X Cell) for 10 minutes at 4° C. followed by membrane staining for 15 min at 4° C. using a combination of FITC anti-CD44 (IM7), V500 anti-CD4 (RM4-5), PE-Cy7 anti-CD8α (53-6.7) and BV421 anti-PD-1 (29F.1A12). Second, cells were fixed and permeabilized using eBioscience Fixation/permeabilization (Thermo Fischer) according to the manufacturer's instructions. Permeabilized cells were then blocked with anti-CD16/CD32 antibody (2.4G2, Bio X Cell) for 15 minutes at 4° C. followed by intracellular staining performed in eBioscience permeabilization buffer (eBioscience) for 30 minutes at 4° C. using PE anti-Foxp3 (FJK-16a) and APC anti-IFNγ (XMG1.2).

In both case, dead cells were excluded using the LIVE/DEAD Fixable Near-IR Dead Cell Stain Kit (Life Technologies). Data were collected on a LSR-II Fortessa flow cytometer and further analyzed using FlowJo software (Tree Star). All antibodies were purchased from BioLegend unless stated otherwise.

### IHC Analysis of Muscle Section

Male C57BL/6 mice were injected in the left tibialis anterior muscle with 10¹⁰ viral genomes (vg) of rAAV1 encoding mOVA-GFP under the muscle-specific SPc5-12 promotor and simultaneously injected or not i.v. with 1×10¹⁰ vg rAAV8 encoding mOVA-GFP under the liver-specific promotor hAAT. After sacrifice at day 14, frozen sections of muscles were prepared and stained for GFP revealed in green, for Major Histocompatibility Class II (MHCII) molecules in red using MAb anti-mouse MHC Class II M5/114 (Bio X Cell) detected by Alexa Fluor® 647 labeled goat anti-Rat antibody (Abcam) and nuclei DAPI staining in blue. Representative sections have been recorded with Leica SP8 confocal imaging station with a 40×objective and a field size of 200 μm×200 μm.

### Anti-OVA IgG ELISA

ELISA microtiter plates (Nunc.) were coated overnight with 50 μl per well of a 10 μg/ml dilution of OVA protein (Sigma-Aldrich) in carbonate buffer pH 9.5. Plates were then washed 3 times with 1×PBS 0.05% Tween and blocked for 2 hours with blocking buffer: 1×PBS 2% BSA at room temperature and washed 3 times. Serial dilutions of experimental sera, as well as of a reference serum from mice immunized with OVA protein emulsified in incomplete Freund adjuvant, were prepared in blocking buffer and incubated in 96 wells plates for 1 hour at 37° C. Then, the plates were washed 2 times and bound anti-OVA IgG were incubated with 1004 of biotinylated horse antimouse IgG (Vector Laboratories, Eurobio, Les Ulis, France) diluted 1/4000 in blocking buffer. Plates were then washed 2 times and incubated with 100 μL/well of Horseradish peroxidase avidin (Vector Laboratories, Eurobio, Les Ulis, France) diluted 1/4000 in blocking buffer for 30 minutes at room temperature and washed again 3 times. Finally, anti-OVA IgG was revealed with TMB substrate reagent set (BD Biosciences). The reaction was stopped after 3-5 minutes with 50 μL/well of H2SO4 2N and the absorbance at 450 nm was determined. Antibodies levels are represented as a ratio of sample dilution over the reference serum dilution corresponding to the same optical densities, considering a linear range in standard curve.

### Statistical Analysis

All data are shown as mean±SEM. For all statistical analyses, Mann-Whitney tests were performed. Data were considered significant when p values were <0.05, with * p<0.05, ** p<0.01, *** p<0.001 and **** p<0.0001 and nonsignificant (ns) when p values were >0.05.

### Results

### Transgene-Specific Immune Tolerance is Established by Dual Muscle-Liver Transduction

In order to establish that rAAV liver transduction promotes immune tolerance towards muscle transgene engraftment, the inventors choose a model transgene encoding fora membrane form of ovalbumin (mOVA), reported to be highly immunogenic after rAAV gene transfer in muscle. For that, two vectors were designed: a muscle-tropic rAAV1 vector encoding for mOVA under the muscle-specific promotor SPc5-12 and a liver-tropic rAAV8 vector encoding for the same mOVA transgene under the liver-specific promoter hAAT (FIG. 2). As expected, rAAV1/mOVA vector intramuscular (im) injection induced a strong anti-OVA CD8⁺ Kb/OVA257 Tetramer⁺ (Tetramer⁺) T cell response in blood at d14 and d28 post injection (FIG. 2A-2B), resulting in a significant decrease in OVA expression in muscle by d29 (FIG. 2D), indicative of immune-related transgene rejection. Of note, analysis of liver lymphocyte populations evidenced an enriched proportion of anti-OVA CD8⁺T cells compared to blood, a result consistent with a previous report showing that activated CD8⁺T cells can accumulate in the liver independently of antigen recognition. These transgene-specific CD8⁺ T cell responses are associated with humoral responses to OVA (FIG. 2C). Taken together, these results show that rAAV1/mOVA muscle targeting induced cellular and humoral responses associated with transgene rejection.

In parallel, we assessed the capacity of the liver-tropic rAAV8/mOVA vector to alleviate transgene immune responses and rejection and found that caudal vein intravenous (i.v.) injection of 10¹⁰ vg rAAV8/mOVA induced neither anti-OVA tetramer⁺ CD8⁺ T cell response in blood at d14 and d28 nor anti-OVA antibody responses (not shown). Liver mOVA expression was confirmed by RT-qPCR under these conditions (not shown), attesting long term-acceptance of the transgene product in accordance with the lack of cellular and humoral responses to the transgene. Next, using concurrent i.m. and i.v. injections of the muscle-tropic rAAV1 and liver-tropic rAAV8 vectors respectively, we found no anti-OVA CD8⁺ T cell response in blood at d14 and d28 (FIGS. 2A-2B) and in liver lymphocyte populations at d29 (Figure S2), as well as no OVA antibody responses (FIG. 2C). Maintenance of transgene expression was effective in both muscle (FIG. 2D) and liver (data not shown) attesting lack of immune rejection. Next, we evaluated the specificity of this suppression and injected rAAV1/mOVA to target the muscle concurrently with an irrelevant rAAV8/hAAT—hFIX to target the liver and evidenced cellular and humoral responses against OVA similar to the i.m. only rAAV1/mOVA injection condition (FIG. 8A-C). Here, the presence of dissimilar transgenes in muscle and liver led to complete rejection of the mOVA transgene in muscle (FIG. 8D) with full acceptance of the hFIX transgene in the liver (not shown). This result indicates that the tolerance induction process requires dual expression of the same transgene in muscle and liver. Of note, we observed no reduction in the humoral responses to the rAAV1 and rAAV8 capsids with dual muscle-liver transduction protocol (data not shown). We also assessed the eventual tolerizing effect of rAAV1/mOVA leakage from the blood circulation to the liver and found that rAAV1/mOVA injection performed using the i.v. route was unable to confer immune protection for muscle transduction (data not shown), indicating that actual rAAV8/hAAT-mOVA liver targeting is mandatory to promote transgene-specific tolerance. To complement these results, we engineered a second set of rAAV vectors encoding for a mOVA-GFP construct, where the full length eGFP protein is fused after the transmembrane part of mOVA (FIG. 1), and found similar requirements for dual muscle-liver expression of the same transgene to achieve muscle transgene engraftment (FIG. 9). Of note, the mOVA-GFP construct harbors a MHC class II epitope, which leads to the induction of detectable IFNγ-producing CD4⁺T cells and enhances OVA-specific antibody responses in mice initially primed with the corresponding GFP peptide (FIG. 9C). In conclusion, dual muscle and liver targeting with rAAV vectors is instrumental in eliminating the occurrence of cellular and humoral immune responses to the transgene, and in allowing transgene expression in muscle.

We then explored the sustainability and robustness of this transgene-specific tolerance and injected the rAAV8/mOVA vector via the i.v. liver route before challenging the mice i.m. at d7 or d28 with rAAV1/mOVA (FIG. 3A). In both cases, we detected no humoral (data not shown) and limited cellular responses (FIG. 3B), and OVA expression was significantly maintained in muscle compared to the control with rAAV1/mOVA i.m. injection alone (FIG. 3C). To assess the robustness of this peripheral tolerance to mOVA, we challenged a second set of mice at d7 or d28 with rAAV1/mOVA-GFP i.m. injections (FIG. 10A), which harbors an immunoreactive MHC class II epitope within the GFP sequence able to prime a CD4⁺T cell response (FIG. 9C). As before, we detected no humoral (data not shown) and limited cellular responses (FIG. 10B), and sustained OVA-GFP expression in muscle (FIG. 10C). Thus, this immune tolerance associated to liver transgene expression appears robust and long-lived even using a muscle transgene harboring an additional MHC II epitope.

Robust Tolerance Induction and Lack of Local Inflammation in Muscle Following Dual Muscle and Liver Transduction of mOVA-GFP Transgene

In order to visualize the level of expression of the transgene and of local inflammation in muscles, we injected rAAV1/mOVA-GFP to target the muscle concurrently or not with rAAV8/mOVA-GFP to target the liver and evidenced GFP staining and local attraction of inflammatory cells via the presence of MHCII positive cells and nuclei staining. High level of inflammatory cells is detected in close apposition to OVA-GFP positive muscle fibers in the muscle only rAAV1/mOVA-GFP injection condition (FIG. 11A), while muscle fibers are intensively and entirely positive for mOVA-GFP without inflammatory cells in the dual muscle rAAV1/mOVA-GFP and liver rAAV8/mOVA-GFP injection condition (FIG. 11B). Hence, together with the results of FIG. 8, these results show that dual muscle and liver transduction leads to complete and robust immune tolerance of mOVA-GFP transgene in muscle, provided that dual expression of the same transgene is achieved in muscle and liver.

### Induction of Immune Tolerance and of Exhausted OVA-Specific CD8⁺T Cells in Presence of Pre-Existing Immunity

Having established that dual muscle-liver transduction allows muscle transgene engraftment, we assessed whether a pre-existing immune response against the transgene product impairs the induction of transgene-specific tolerance. For that, mice were pre-immunized or not with OVA protein emulsified in incomplete Freund's adjuvant (IFA) at d0 and injected at day 14 with either single i.m. rAAV1/mOVA or dual i.m. rAAV1/mOVA and i.v. rAAV8/mOVA injections. As expected, OVA/IFA immunization was particularly effective to prime a humoral anti-OVA response monitored after i.m. rAAV1/mOVA injection (FIG. 4B). Importantly, we found that dual i.m. rAAV1/mOVA and i.v. rAAV8/mOVA injections reduced to very low levels OVA-specific CD8⁺T cells responses and humoral responses (FIGS. 4A-B) and ensured long-term OVA expression in muscle (FIG. 4C), despite occurrence of preexisting immunity. Thus, the transgene-specific tolerance imposed by dual muscle-liver transduction overrides both emerging and memory CD8⁺T cell as well as preexisting antibody responses to the transgene product.

As shown above, dual muscle-liver transduction is able to prevent adverse responses associated with preexisting transgene-specific immunity present in the host. Under these conditions, we noticed the presence of a residual fraction of transgene-specific CD8⁺T cells in the spleen of mice initially primed with OVA/IFA and receiving i.v. rAAV8/mOVA injections (data not shown). As OVA/IFA pre-immunization did not compromise transgene muscle expression (FIG. 4C), we analyzed the phenotype of residual OVA-specific CD8⁺T cells present after OVA/IFA or OVA257 peptide/IFA immunization followed by dual muscle-liver transduction (FIG. 5). The OVA257 peptide/IFA injection condition was used to visualize the fate of transgene-specific CD8⁺ T cells independently of CD4⁺T cell and B cell priming. In these later two pre-immunization conditions, dual muscle-liver transduction reduced significantly the quantity of OVA-specific CD8⁺T cells compared to single muscle rAAV transduction, with a residual fraction of OVA-specific CD8⁺T cells present in both cases (FIG. 5A-B). Of high interest, we found that these residual OVA-specific CD8⁺T cells expressed higher levels of PD-1 (FIGS. 5A-5C) and slightly higher levels of CD44 and CD8 in comparison with those generated after muscle transduction alone (FIG. 5C), raising the possibility of a conversion of OVA-specific CD8⁺ effector T cells into exhausted CD8⁺T cells.

To qualify the functional ability of these residual OVA-specific CD8⁺T cells, we assayed their ability to produce INFy in response to MHC I restricted OVA257 peptide stimulation in conjunction with PD-1 surface expression (FIG. 6). As evidenced by intracellular staining, muscle only transduction with i.m. rAAV1/mOVA generated mostly OVA-specific CD8⁺T cells with high INFy production and lower PD-1 surface expression, whereas dual muscle-liver transduction with i.m. rAAV1/mOVA and i.v. rAAV8/mOVA injections generated mostly residual OVA-specific CD8⁺T cells with no INFy production capability (FIG. 6A). Upon quantification, no INFy production was observed after simultaneous i.m. rAAV1/mOVA and i.v. rAAV8/mOVA injection in both OVA/IFA and OVA257/IFA pre-immunization conditions (FIG. 6B). Consequently, the residual OVA-specific CD8⁺T cells expressing high level of PD-1 observed after dual muscle-liver transduction (FIG. 5) lack INFγ production capacity and correspond to typically exhausted CD8⁺T cells. Moreover, OVA muscle transgene engraftment was effective after dual i.m. rAAV1/mOVA and i.v. rAAV8/mOVA injections, independently of pre-immunization (FIG. 6C). These results demonstrate that dual muscle-liver transduction is sufficient to protect muscle OVA expression even in presence of a pre-existing CD8⁺ T cell immunity induced by OVA/IFA and OVA257 peptide/IFA, through a mechanism implying in part the generation of CD8⁺T cells exhibiting a typically exhausted phenotype.

Transgene-Specific CD8⁺T Cell Tolerance is Established Despite Preexisting CD4⁺T Cell responses.

Last, as preexisting CD4⁺T cell responses to dystrophin have been observed in DMD patients 24, 25, we wondered whether a preexisting OVA-specific CD4⁺T cell response could influence the level of transgene-specific tolerance achieved after dual muscle-liver targeting. For that, mice were first immunized with the MHC class II-restricted OVA323 epitope before being injected with either rAAV1/mOVA i.m. or dual rAAV1/mOVA i.m. and rAAV8/mOVA i.v. As expected, we found that OVA323 immunization significantly primed INFγ production in activated CD4+CD44^{hi}T cells generated after single i.m. rAAV1/mOVA (FIG. 7A), and anti-OVA humoral response (FIG. 7B). In dual i.m. rAAV1/mOVA and i.v. rAAV8/mOVA injected mice, INFγ production was equally detected in activated CD4⁺CD44^{hi}T cells (FIG. 7A) without compromising muscle OVA expression (FIG. 7D), in accordance with the lack of OVA257 tetramer⁺ CD8⁺T cell and humoral response observed under these conditions (FIG. 7B-C). Altogether, although OVA323 immunization was effective to boost antibody production (FIG. 7B) after i.m. injection only, dual i.m. and i.v. injections were operant in controlling humoral and cellular CD8⁺T, but not CD4⁺T cell responses to the transgene, and this was sufficient to protect muscle OVA expression. The results demonstrate that transgene-specific CD8⁺T cell tolerance is established despite preexisting CD4⁺T cell responses, which persisted after dual muscle-liver transduction.

### CONCLUSION

No prior studies addressed the influence of concurrent muscle and liver transduction on the induction of transgene-specific tolerance for muscle applications and no studies addressed under these circumstances whether preexisting immunity is deleterious for muscle transgene engraftment.

Surprisingly, the inventors found that dual muscle and hepatocyte OVA expression led to complete absence of OVA-specific CD8⁺T cells in the liver, blood and spleen. Furthermore, the inventors observed an accumulation of OVA-specific CD8⁺T cell in liver after single muscle injection with rAAV1/SPc5-12-mOVA muscle-specific vector, reflecting a transient accumulation of activated CD8⁺T cells occurring in liver independently of local antigen expression.

These results indicate that activated OVA-specific CD8⁺T cells generated by muscle transduction gain access to the liver tissue where secondary cognate interactions with OVA antigen can take place, leading to either disposal and/or functional inactivation of OVA-specific CD8⁺T cells. Indeed, CD8⁺T cells have been shown to be disposed in the liver following antigen recognition through direct capture and internalization by hepatocyte in a mechanism referred to as suicidal emperipolesis.

The results in the context of dual muscle-liver rAAV targeting demonstrate two outcomes for endogenous CD8⁺T cells depending on the initial state of the host immune system with respect to the transgene. When mice are naïve to the transgene, transgene-specific CD8⁺T cells are absent from all tissues tested. When mice are primed to induce preexisting immunity to the transgene, the inventors observed a massive reduction of OVA-specific CD8⁺T cells in the spleen, with a remaining fraction of OVA-specific CD8⁺T cells expressing high levels of PD-1 and somewhat higher expression of CD44, a feature found in exhausted CD8⁺T cells but insufficient for a clear demonstration of their state. Assaying the function of these OVA-specific CD8⁺T cells, the inventors found that these PD-1^{hi} CD8⁺T cells did not produce IFNγ in response to antigen stimulation, a result which correlates with persistent transgene expression in muscle and advocates for their status of exhausted CD8⁺T cells.

Further, the results demonstrate here that processing of a defined muscle transgene by antigen presenting cells of the host leads to CD8⁺T cells responses, which are tolerized after recognition of hepatocyte-expressed, host MHC class I transgene complexes, even in presence of preexisting immunity.

Monitoring the humoral response to the transgene, the study extends also to muscle-associated transgenes. Humoral responses to the transgene were drastically reduced after dual muscle-liver transduction but liminal levels of anti-OVA antibodies were nevertheless detected after tolerance induction in recipients preimmunized with OVA protein, but not with OVA257 peptide.

Assessing the potential of OVA-specific CD4⁺T cell responses on anti-OVA antibody production, the inventors observed that preimmunization with the MHC class II-restricted OVA epitope OVA323 peptide is effective to prime CD4⁺T cells and enhances anti-OVA antibody production after rAAV/mOVA muscle transfer, ascertaining the role of CD4⁺T cell responses in humoral immunity to the transgene.

Exploring the fate of transgene-specific CD4⁺T cells generated after OVA323 peptide immunization, we found that dual muscle-liver transduction led to detectable anti-OVA323 IFNγ producing CD4⁺T cells but to barely detectable levels the anti-OVA antibody response. Thus, of high interest for clinical situations where preexisting immunity to the transgene is encountered, the presence of anti-OVA CD4⁺T cell responses did not impair the induction of CD8⁺T cell and humoral tolerance. This result is compatible with a qualitative alteration of CD4⁺T cells and with the fact that liver-based tolerance induction to FIX confers transferable tolerogenic properties to the CD4⁺T cell compartment. Here, the model transgene system allowed to recapitulate the effectiveness of liver-based tolerance induction counteracting preexisting immune responses in multiple situations. Both preexisting humoral responses and CD8⁺ and CD4+T cells responses were found impacted by dual muscle-liver transgene expression, with transgene-specific CD8⁺T cells undergoing retention and/or depletion and exhaustion, and transgene-specific CD4⁺T cells remaining present but unable to boost antibody production. These CD4⁺T cells are presumably forming a pool of cells able to undergo conversion into Foxp3⁺Treg cells, as evidenced in multiple sclerosis models.

Overcoming muscle immune response to therapeutic transgenes is of importance in the treatment of muscular dystrophies. Muscle monogenic disorders can induce tissue inflammation and particularly in Duchenne's muscular dystrophy patients, where contraction-induced damages release cytoplasmic content that can stimulate innate immunity, promote chronic muscle inflammation and worsen adverse immune responses to the therapeutic transgene. In this context, concurrent delivery of the transgene in muscle and liver is relevant to cope with adverse immune responses due to preexisting immunity.

### REFERENCES

Bowen DG, Zen M, Holz L, Davis T, McCaughan GW, Bertolino P. The site of primary T cell activation is a determinant of the balance between intrahepatic tolerance and immunity. J Clin Invest. September;114(5):701-12. (2004).
Ghenassia, A. Gross DA, Lorain S, Tros F, Urbain D, Benkhelifa-Ziyyat S, Charbit A, Davoust J, Chappert P. Intradermal Immunization with rAAV1 Vector Induces Robust Memory CD8(+) T Cell Responses Independently of Transgene Expression in DCs. Molecular therapy 25, 2309-2322 (2017).
Gross, D. A. Gross DA, Ghenassia A, Bartolo L, Urbain D, Benkhelifa-Ziyyat S, Lorain S, Davoust J, Chappert P. Cross-Presentation of Skin-Targeted Recombinant Adeno-associated Virus 2/1 Transgene Induces Potent Resident Memory CD8(+) T Cell Responses. Journal of virology 93 (2019).
Vidal P, Pagliarani S, Colella P, Costa Verdera H, Jauze L, Gjorgjieva M, Puzzo F, Marmier S, Collaud F, Simon Sola M, Charles S, Lucchiari S, van Wittenberghe L, Vignaud A, Gjata B, Richard I, Laforet P, Malfatti E, Mithieux G, Rajas F, Comi GP, Ronzitti G, Mingozzi F. Rescue of GSDIII Phenotype with Gene Transfer Requires Liver- and Muscle-Targeted GDE Expression. Mol Ther. March 7;26(3):890-901. (2018).

### REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “APIC-65194-Sequence-Listing_ST25.txt”, created Nov. 12, 2021, file size of 4,096 bytes, is hereby incorporated by reference.

1. A combination of A method of treatment of a muscular dystrophy in a subject comprising an immune system, the method comprising:
administering to the subject
i. a first recombinant adeno-associated viral (rAAV) vector comprising a capsid and a cassette comprising a 5′ ITR sequence, a liver-specific promotor, a nucleic acid sequence coding for a protein product to be tolerated by the immune system, and a poly A chain, and
ii. a second rAAV vector comprising a capsid and a cassette comprising a 5′ ITR sequence, a muscle-specific promotor, a nucleic acid sequence coding for the protein product to be tolerated by the immune system of the first rAAV vector, and a poly A chain,
wherein the first rAAV vector targets a liver of the subject and the second rAAV vector targets muscle tissues of the subject.

2. The method of treatment according to claim 1, wherein the nucleic acid sequences coding for the protein product to be tolerated by the immune system code for a cell-associated protein product.

3. The method of treatment according to claim 2, wherein the cell-associated protein product is a protein product delivered to the cytosol or a transmembrane protein product.

4. The method of treatment according to claim 1, wherein the nucleic acid sequences coding for the protein product to be tolerated by the immune system code for a protein product comprising an epitope recognized by T-cells or B-cells.

5. The method of treatment according to claim 1, wherein the administration of the first and second rAAV vectors eliminates or attenuates the occurrence of cellular and humoral immune responses to the protein product, allowing said protein product to be tolerated by the immune system of the subject and/or its expression in muscle of the subject.

6. The method of treatment according to claim 1, wherein the subject presents a preexisting immunity towards the protein product to be tolerated by the immune system of the subject.

7. The method of treatment according to claim 1, wherein the liver-specific promotor of the first rAAV vector is a hepatocyte-specific promotor (hAAT).

8. The method of treatment according to claim 1, wherein the capsids of the first and second rAAV vectors are selected from the group consisting of AAV1, AAV2, AAV3, AAV4, AAV5, AAV6, AAV7, AAV8, AAV9, AAV10, AAV11, and any combination thereof.

9. The method of treatment according to claim 8, wherein the capsid of the first rAAV vector is an AAV8 capsid.

10. The method of treatment according to claim 1, wherein the protein product to be tolerated by the immune system is a muscle specific protein or a neuromuscular protein.

11. The method of treatment according to claim 1, wherein the nucleic acid sequences coding for a protein product to be tolerated by the immune system are coding for a peptide selected from a sequence of the group consisting of sequences of microdystrophin constructs, Emerin, Lamin A/C, Spectrin repeat containing, nuclear envelope 1 (nesprin 1), Spectrin repeat containing, nuclear envelope 2 (nesprin 2), Transmembrane protein 43, Torsin A interacting protein 1, Double homeobox 4, Structural maintenance of chromosomes flexible hinge domain containing 1, Polymerase I and transcript release factor(M), Myotilin, Caveolin 3, HSP-40 homologue, subfamily B, number 6, Desmin, Transportin 3, Heterogeneous nuclear ribonucleoprotein D-like, Calpain 3, Dysferlin, Gamma sarcoglycan, Alpha sarcoglycan, Beta sarcoglycan, Delta-sarcoglycan, Telethonin, Tripartite motif-containing 32, Fukutin-related protein, Titin, Protein-O-mannosyltransferase 1, Anoctamin 5, Protein-O-mannosyltransferase 2, O-linked mannose beta1,2-N-acetylglucosaminyltransferase, Dystroglycan1, plectin, Desmin, trafficking protein particle complex 11, GDP-mannose pyrophosphorylase B, Isoprenoid synthase domain containing, Acid alpha-glucosidase preproprotein, LEVI and senescent cell antigen-like domains 2, blood vessel epicardial substance, Torsin A interacting protein 1, Protein 0-Glucosyltransferase 1, Dolichyl-phosphate mannosyltransferase polypeptide 3, Valosin-containing protein, and plectin.

12. The method of treatment according to claim 11, wherein the muscular dystrophy is Duchene Muscular Dystrophy (DMD) and the protein product to be tolerated by the immune system is a microdystrophin construct.

13. The method of treatment according to claim 1, wherein the first rAAV vector is administered intravenously and the second rAAV vector is administered intramuscularly to the subject.

14. The method of treatment according to claim 1, wherein the first rAAV vector is administered before the second rAAV vector.

15. A pharmaceutical composition for use in treating a muscular dystrophy comprising:
i. a first recombinant adeno-associated viral (rAAV) vector comprising a capsid and a cassette comprising a 5′ ITR sequence, a liver-specific promotor, a nucleic acid sequence coding for a protein product to be tolerated by the immune system, and a poly A chain, and
ii. a second rAAV vector comprising a capsid and a cassette comprising a 5′ ITR sequence, a muscle-specific promotor, a nucleic acid sequence coding for the protein product to be tolerated by the immune system of the first rAAV vector, and a poly A chain.

16. A kit of parts for use in treating a muscular dystrophy comprising:
i. a first recombinant adeno-associated viral (rAAV) vector comprising a capsid and a cassette comprising a 5′ ITR sequence, a liver-specific promotor, a nucleic acid sequence coding for a protein product to be tolerated by the immune system, and a poly A chain, and
ii. a second rAAV vector comprising a capsid and a cassette comprising a 5′ ITR sequence, a muscle-specific promotor, a nucleic acid sequence coding for the protein product to be tolerated by the immune system of the first rAAV vector, and a poly A chain.

17. The kit of parts according to claim 16, wherein the first and second rAAV vectors are provided in unit dosage forms.

18. The kit of parts according to claim 16, further comprising a means for testing or detecting a preexisting immunity toward the protein product to be tolerated by the immune system.

19. The method of treatment according to claim 1, wherein:
the administration of the first and second rAAV vectors comprises repeated administration of at least one of the first or second rAAV vectors; and/or
the administration of the first rAAV vector is separated in time from the administration of the second rAAV vector.

20. The method of treatment according to claim 1, wherein the method further comprises a step of testing for and/or detecting the presence of a preexisting immunity toward the protein product to be tolerated by the immune system.
